(19) United States
(12) Patent Application Publication
(10) Pub. No.: US 2024/0127904 A1
(43) Pub. Date: Apr. 18, 2024

(54) COVID MULTI-BIOME

(71) Applicant: Microbiota I - Center (Magic) Limited, Hong Kong SAR (CN)

(72) Inventors: Siew Chien NG, Hong Kong SAR (CN); Ka Leung Francis CHAN, Tai Po (CN); Qin LIU, Ma On Shan (CN)

(21) Appl. No.: 18/213,136

(22) Filed: Jun. 22, 2023

Related U.S. Application Data
(60) Provisional application No. 63/355,443, filed on Jun. 24, 2022.

(51) Int. Cl. G16B 20/00; A61K 35/741; A61K 35/745; A61K 45/06; C12Q 1/6869; C12Q 1/70; G16B 30/00; G16B 40/20; G16H 10/40; G16H 50/30

(52) U.S. Cl. CPC G16B 20/00; A61K 35/741; A61K 35/745; A61K 45/06; C12Q 1/6869; C12Q 1/701; G16B 30/00; G16B 40/20; G16H 10/40; G16H 50/30; A61K 2035/115

(57) ABSTRACT

The present invention relates to methods for assessing one's risk for suffering from severe COVID or long COVID symptoms. Also provided are methods for reducing the risk of developing severe COVID and/or long COVID symptoms.

### RELATED APPLICATIONS

This application claims priority to U.S. Provisional Patent Application No. 63/355,443, filed Jun. 24, 2022, the contents of which are hereby incorporated in the entirety for all purposes.

### BACKGROUND OF THE INVENTION

In recent years, viral and bacterial infection is becoming more prevalent worldwide and presents a serious public health threat. For example, the Coronavirus-2019 (COVID-19) global pandemic of a respiratory disease caused by severe acute respiratory syndrome coronavirus 2 (SARS-CoV-2) has affected over 520 million people worldwide, including over 6 million deaths, and is exacerbated by a lack of effective therapeutics that have received official approval as well as a lack of proven safe and effective vaccines capable of offering protection against a broad spectrum of viral variants. Patients with SARS-CoV-2 infection can experience a range of clinical manifestations, from no symptoms to critical illness. Up to three-quarters of patients experienced at least one symptom at 6 months after recovering from COVID-19, a phenomenon known as post-acute COVID-19 syndrome (PACS). Even COVID-19 patients with mild disease or minimal symptoms may experience PACS that can be debilitating affecting different systems including the lung, heart, gut, musculoskeletal, brain etc. Thus, there is an urgent need for identifying individuals at risk of severe COVID-19 and PACS for early and timely management.

### BRIEF SUMMARY OF THE INVENTION

The present inventors discovered in their studies that certain gut microbial species and certain clinical parameters can be used to assess the presence or risk of severe COVID or post-acute COVID syndrome (PACS, or long COVID) in individuals who may or may not have been diagnosed of COVID-19. Further, certain gut microbial species and certain clinical parameters can be used to determine a COVID patient's viral shedding duration. The gut microorganisms so identified in this study can serve to support new methods and compositions as an integral part of the COVID-19 risk assessment, therapeutic and/or prophylactic treatment, and long-term management.

In a first aspect, the present invention provides a method for determining the presence or risk of severe COVID or PACS in a subject. The method includes these steps: (1) obtaining a set of training data by determining in fecal samples the relative abundance of the bacteria, viral, and fungi species listed in Table 3 and the clinical factors listed in Table 3 obtained from a cohort of subjects who suffer from severe COVID-19 or PACS as well as from another cohort of subjects who does not suffer from severe COVID-19 or PACS; (2) determining the relative abundance of the species and clinical factors listed in Table 3 in the patient; (3) comparing the relative abundance of the species and clinical factors listed in Table 3 obtained from step (2) from the patient with the training data using random forest model, wherein decision trees are generated by random forest from the training data, and wherein the relative abundance of the species and clinical factors listed in Table 3 obtained in step (2) from the patient are run down the decision trees to generate a risk score; and (4) determining the patient as having or at increased risk for severe COVID-19 or PACS when the risk score is greater than 0.5, and determining the patient as not having or at no increased risk for severe COVID-19 or PACS when the risk score is no greater than 0.5. In some embodiments, the patient being assessed has been diagnosed with COVID, although he may or may not exhibit any symptoms for the disease. In some embodiments, the patient has not been diagnosed with COVID, but the patient may be at elevated risk for COVID (for example, due to his professional activities) or have had a known event exposing him to the disease (for example, had been in close contact with someone who suffers from COVID within the past 2-3 days). In some embodiments, each of steps (1) and (2) comprises determining the level of a DNA, RNA, or protein unique to one or more of the bacterial species set forth in Table 3. In some embodiments, each of steps (1) and (2) comprises metagenomics sequencing. In some embodiments, each of steps (1) and (2) comprises a polymerase chain reaction (PCR), for example, a quantitative PCR (qPCR). In some embodiments, the claimed method further comprises treating the patient who has been determined as having or at increased risk for severe COVID-19 or PACS to prevent or alleviate symptoms of severe COVID-19 or PACS. In some embodiments, the treating step comprises administering to the patient a composition comprising an effective amount of (a) Bifidobacterium adolescentis or Faecalibacterium prausnitzii, or (b) an inhibitor specifically suppressing Ruminococcus gnavus, Klebsiella species (Klebsiella quasipneumonia, Klebsiella pneumoniae, and Klebsiella variicola), Clostridum species (Clostridum bolteae and Clostridium innocuum and Clostridium spiroforme); Asperigillus flavus, Candida glabrata, Candida albucans; Mycobacterium phage MyraDee, Pseudomonas virus Pf1, or Klebsiella phage. Optionally, one or more additional therapeutic agents known to be used for treating COVID, such as those named in this disclosure, may be also administered to the patient, e.g., for the symptoms of severe or long COVID during the time period when the patient exhibits one or more of such symptoms. In some embodiments, the treating step comprises fecal microbiota transplantation (FMT), for example, by way of delivery to the small intestine, ileum, or large intestine of the patient a composition comprising processed donor fecal material. In some embodiments, the composition is formulated for oral administration, e.g., in the form of a food or beverage item. In some embodiments, the composition is formulated for direct deposit to the patient's gastrointestinal tract.

In the second aspect, the present invention provides a method for predicting the virus SARS-CoV2 shedding duration in a COVID-19 patient. The method includes these steps: (1) obtaining a set of training data by determining in fecal samples the relative abundance of species and clinical factors listed in Table 4 in a cohort of subjects who have been diagnosed with COVID-19 and have had their SARS-CoV-2 viral shedding duration analyzed and determined; (2) determining the relative abundance of the species and clinical factors listed in Table 4 in the COVID-19 patient; (3) comparing the relative abundance of species and clinical factors listed in Table 4 in the subject with the training data using random forest model; and (4) generating viral shedding duration by the random forest model. In some embodiments, steps (1) and (2) each comprises determining the level of a DNA, RNA, or protein unique to one or more of the bacterial species set forth in Table 4. In some embodiments, steps (1) and (2) each comprises metagenomics sequencing. In some embodiments, steps (1) and (2) each comprises a polymerase chain reaction (PCR), such as a quantitative PCR (qPCR). In some embodiments, the method further comprises a step of keeping the patient in isolation for the viral shedding duration determined in step (4). In some embodiments, the claimed method further comprises treating the patient who has been diagnosed with COVID-19 and remained in isolation for the duration of predicted time duration of SARS-CoV2 virus shedding for the symptoms of COVID and/or causes of the symptoms. In some embodiments, the treating step comprises administering to the patient a composition comprising an effective amount of (a) Bifidobacterium adolescentis or Faecalibacterium prausnitzii, or (b) an inhibitor specifically suppressing Ruminococcus gnavus, Klebsiella species (Klebsiella quasipneumonia, Klebsiella pneumoniae, and Klebsiella variicola), Clostridum species (Clostridum bolteae and Clostridium innocuum and Clostridium spiroforme); Asperigillus flavus, Candida glabrata, Candida albucans; Mycobacterium phage MyraDee, Pseudomonas virus Pf1, or Klebsiella phage. Optionally, one or more additional therapeutic agents known to be used for treating COVID, such as those named in this disclosure, may be also administered to the patient, e.g., for the predicted time duration of viral shedding and/or required isolation. In some embodiments, the treating step comprises fecal microbiota transplantation (FMT), for example, by way of delivery to the small intestine, ileum, or large intestine of the patient a composition comprising processed donor fecal material. In some embodiments, the composition is formulated for oral administration, e.g., in the form of a food or beverage item. In some embodiments, the composition is formulated for direct deposit to the patient's gastrointestinal tract.

### BRIEF DESCRIPTION OF THE DRAWINGS

FIG. 1 Schematic diagram of study design.

FIG. 2A-FIG. 2H Integration of gut multi-biome data through weighted similarity network fusion (WSNF) approach. (FIG. 2A) Schematic overview of the study design, depicting the total number of samples and participants from whom data were available. (FIG. 2B) Heatmap illustrating pairwise patient WSNF similarity scores stratified by spectral clustering (cluster 1, n=63; cluster 2, n=70) according to integrated multi-biome profiles, derived from n=133 biologically independent samples. (FIG. 2C) MaAslin analysis of observed clusters illustrating discriminant taxa at baseline. (FIG. 2D) Symptoms of COVID-19 patients between two identified patient clusters. The proportion of diarrhea, chills, headache, fever and cough at cluster 1 were significantly higher than cluster 2 (chi-square test with one degree of freedom, FDR correction). (FIG. 2E) Comparison of viral load (copies/mL) (FIG. 2F) Severity of disease (FIG. 2G) C-reactive protein (CRP) concentration (FIG. 2H) CXCL levels between two identified patient clusters.

FIG. 3A-FIG. 3C Prognostic roles of gut integrative microbiomes for post-acute COVID-19 syndrome. (FIG. 3A) Comparison of α-diversity (Shannon diversity index, P=0.029) of patients at 6 months after virus clearance between two identified patient clusters and Principal Coordinate Analysis (PCoA) of gut multi-biome of patients at 6 months after virus clearance based on Bray-Curtis dissimilarity illustrates two patient clusters. (FIG. 3B) MaAslin analysis of observed clusters illustrating discriminant taxa at 6 months after virus clearance. (FIG. 3C) Comparison of post-acute symptoms of COVID-19 patients in two clusters.

FIG. 4A-FIG. 4H Random forest classifier model trained on multi-biome and clinical data can predict the duration of viral shedding for individual COVID-19 patients. (FIG. 4A) The input data is a vector with four parts: demographics, blood test, cytokines and gut multi-biome profile. To estimate model accuracies, a train-test sample split of 70% for training and 30% for testing was utilized. The testing data was then used to estimate the accuracy of the random forest model. (FIG. 4B) Box-and-whisker plot displaying the distribution of the AUC score for the cross-validation on the training set and the AUC score for the single measurement taken on the test set, obtained by random forest classification. (FIG. 4C-FIG. 4G) Top features contribute to differentiating clusters in the random forest models. (FIG. 4H) Integration of multi-biome and clinical data for predicting the duration of viral shedding of SARS-CoV-2. The predicted positive time was paired with the real positive time for accuracy evaluation, and the accuracy was calculated at different error levels from ±0 to ±5 days.

FIG. 5A-FIG. 5F Network analysis of the interactome of COVID-19 patients. (FIG. 5A) Venn diagram summarizing the observed interactions of the multi-biome (FIG. 5B) Summary of the positive or negative association numbers between bacteria, fungi and viruses in two clusters. Visualization of the interactome's negative interactions between the most abundant taxa of bacteria and virus (FIG. 5C), fungi and virus (FIG. 5D), (FIG. 5E) Total and overlapped number of interactions in two clusters. (FIG. 5F) Microbial network graphs in two clusters centring on the Clostridium spiroforme node. Microbes directly interacting with Clostridium spiroforme are coloured to reflect positive (red) or negative (blue) interactions.

FIG. 6A-FIG. 6B Key microbes to maintain network integrity. Network visualization of key taxa in cluster 1 (FIG. 6A) and cluster 2 (FIG. 6B). Coloured circles represent microbes and lines represent their associated interactions. Circle size (degree) reflects the number of direct interactions for a given microbe (termed ‘busy’). Circle border thickness represents calculated stress centrality for each microbe, while colour depth reflects the betweenness centrality (the ‘influence’) of the microbe in the network.

FIG. 7A-FIG. 7E Comparison of gut microbiome composition and microbiome function in different clusters. Comparison of (FIG. 7A) diversity (Shannon index) and PCoA plot (FIG. 7B) illustrating multi-biome diversity in two clusters. (FIG. 7C) Microbiome function profiling in COVID-19 patients. The volcano plot illustrating log 2 fold change between the levels of function between the two clusters. (FIG. 7D) Urea cycle pathway (Log 10 relative abundance) is positively correlated with detected blood urea levels (FIG. 7E) The blood urea level is significant higher in cluster 1.

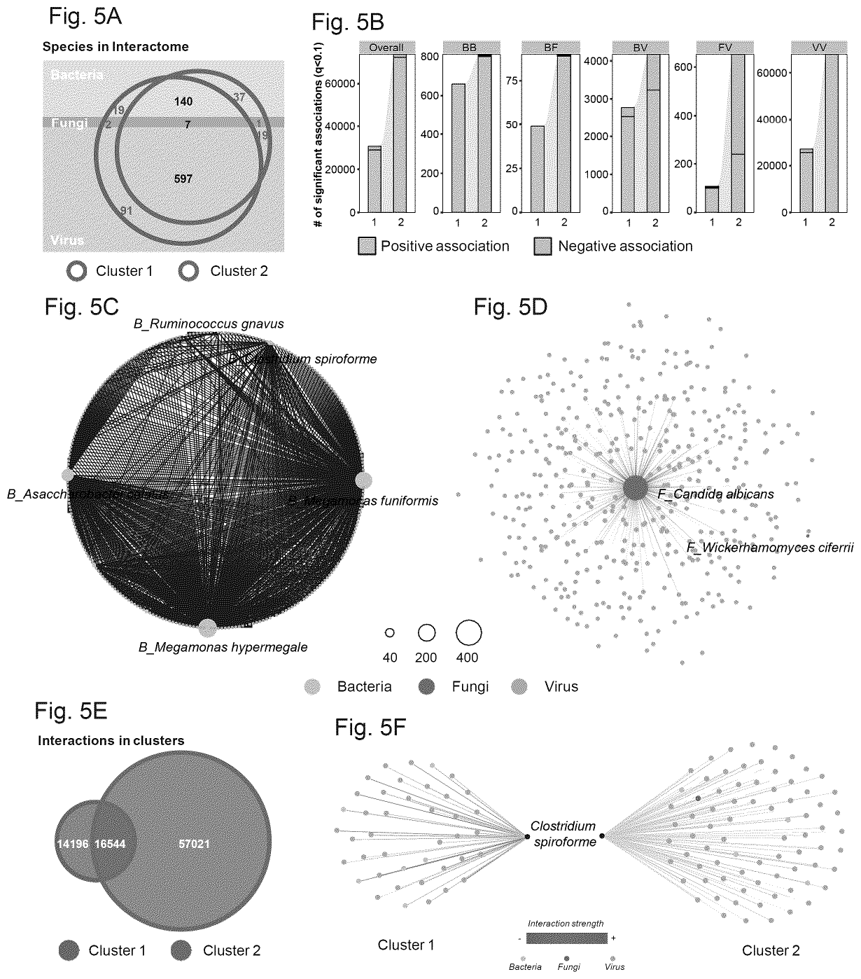

FIG. 5A-FIG. 5C Comparison of positive duration and viral load in respiratory samples and stool samples of COVID-19 patients. (FIG. 8A) Vial shedding duration in respiratory samples and (FIG. 8B) viral load in stool samples of patients within cluster 1 was significantly higher than cluster 2. (FIG. 8C) Positive and negative numbers of SARS-CoV-2 in baseline stool samples detected by RT-PCR from COVID-19 Patients (N=79).

FIG. 9A-FIG. 9D Urea cycle in different clusters. (FIG. 9A) the relative abundance of urea cycle in cluster 1 and cluster 2; (FIG. 9B) the schematic diagram of urea cycle showing the involved enzymes and intermediate metabolites; (FIG. 9C) the relative abundance of involved enzymes in cluster 1 and cluster 2; (FIG. 9D) the contribution of microbes to the relative abundance of K01940 (argininosuccinate synthase).

FIG. 10A-FIG. 10K Comparison of metabolite pathway and detected level in two clusters, and correlations between pathways and detected metabolites of (FIG. 10A-FIG. 10C) acetic, (FIG. 10D-FIG. 10F) L-isoleucine biosynthesis, (FIG. 10G-FIG. 10H) L-isoleucine degradation, and (FIG. 10I-FIG. 10K) L-arginine.

FIG. 11A-FIG. 11D Longitudinal analysis of gut microbiota diversity in patients with COVID-19 at baseline, 3-month follow-up and 6-month follow-up in subjects of cluster 1 (FIG. 11A) and cluster 2 (FIG. 11B). Principal Coordinate Analysis (PCoA) of multi-biome of patients at baseline, 3-month follow-up and 6-month follow-up in subjects of cluster 1 (FIG. 11C) and cluster 2 (FIG. 11D).

FIG. 12A-FIG. 12J Correlations between viral shedding duration and top 10 contributors for prediction model.

FIG. 13A-FIG. 13B Dynamics of interactome from baseline to 3-month follow-up and 6-month follow-up in cluster 1 (FIG. 13A) and cluster (FIG. 13B).

FIG. 14A-FIG. 14D Network analysis of the interactome of COVID-19 patients at 6-month follow-up. (FIG. 14A) Venn diagram summarizing the observed interactions of the multi-biome (FIG. 14B) Summary of the positive or negative association numbers between bacteria, fungi and viruses in two clusters. (FIG. 14C) Total and overlapped number of interactions in two clusters. (FIG. 14D) Characterizations of microbial network in two clusters.

FIG. 15A-FIG. 15B Key microbes to maintain network integrity at 6-month follow-up. Network visualization of key taxa in cluster 1 (FIG. 15A) and cluster 2 (FIG. 15B). Coloured circles represent microbes and lines represent their associated interactions. Circle size (degree) reflects the number of direct interactions for a given microbe (termed ‘busy’). Circle border thickness represents calculated stress centrality for each microbe, while colour depth reflects the betweenness centrality (the ‘influence’) of the microbe in the network.

### DEFINITIONS

As used herein, the term “SARS-CoV-2 or severe acute respiratory syndrome coronavirus 2,” refers to the virus that causes Coronavirus Disease 2019 (COVID-19). It is also referred to as “COVID-19 virus.”

The term “post-acute COVID-19 syndrome (PACS)” or “long COVID” is used to describe a medical condition in which a patient who has recovered from COVID, as indicated by a negative PCR report at least 2 weeks prior (e.g., from at least 3 or 4 weeks earlier), yet continuously and stably exhibits one or more symptoms of the disease without any notable progression, e.g., after a 4-week or longer time period following the initial onset of COVID symptoms. The symptoms may include respiratory (cough, sputum, nasal congestion/runny nose, shortness of breath), neuropsychiatric (headache, dizziness, loss of taste, loss of smell, anxiety, difficulty in concentration, difficulty in sleeping, sadness, poor memory. blurred vision), gastrointestinal (nausea, diarrhea, abdominal pain, epigastric pain), dermatological (hair loss), or musculoskeletal (joint pain, muscle pain) symptoms, as well as fatigue.

The term “severe COVID-19” or “severe COVID” is used to refer to the disease state of a person who has been diagnosed with COVID-19 and has developed one or more of the following symptoms: difficulty breathing (e.g., more than 30 breaths per minute at rest), decreased saturated oxygen level (e.g., under 93%, especially under 90%), elevated heartbeat, persistent high body temperature, pneumonia or pneumonitis, acute respiratory distress syndrome (ARDS), and even death. Typically, although not in all cases, a patient suffering from “severe COVID-19” requires hospitalization. Furthermore, “severe COVID” often refers to the disease state during its acute phase.

The term “inhibiting” or “inhibition,” as used herein, refers to any detectable negative effect on a target biological process, such as RNA/protein expression of a target gene, the biological activity of a target protein, cellular signal transduction, cell proliferation, presence/level of an organism especially a micro-organism, any measurable biomarker, bio-parameter, or symptom in a subject, and the like. Typically, an inhibition is reflected in a decrease of at least 10%, 20%, 30%, 40%, 50%, 60%, 70%, 80%, 90% or greater in the target process or parameter, when compared to a control. “Inhibition” further includes a 100% reduction, i.e., a complete elimination, prevention, or abolition of a target biological process or signal. The other relative terms such as “suppressing,” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal. On the other hand, terms such as “activate,” “activating,” “activation,” “increase,” “increasing,” “promote,” “promoting,” “enhance,” “enhancing,” or “enhancement” are used in this disclosure to encompass positive changes at different levels (e.g., at least about 5%, 10%, 20%, 30%, 40%, 50%, 60%, 70%, 80%, 90%, 100%, 200%, or greater such as 3, 5, 8, 10, 20-fold increase compared to a control level in a target process, signal, or parameter.

As used herein, the term “treatment” or “treating” includes both therapeutic and preventative measures taken to address the presence of a disease or condition or the risk of developing such disease or condition at a later time. It encompasses therapeutic or preventive measures for alleviating ongoing symptoms, inhibiting or slowing disease progression, delaying of onset of symptoms, or eliminating or reducing side-effects caused by such disease or condition. A preventive measure in this context and its variations do not require 100% elimination of the occurrence of an event; rather, they refer to a suppression or reduction in the likelihood or severity of such occurrence or a delay in such occurrence.

The term “severity” of a disease refers to the level and extent to which a disease progresses to cause detrimental effects on the well-being and health of a patient suffering from the disease, such as short-term and long-term physical, mental, and psychological disability, up to and including death of the patient. Severity of a disease can be reflected in the nature and quantity of the necessary therapeutic and maintenance measures, the time duration required for patient recovery, the extent of possible recovery, the percentage of patient full recovery, the percentage of patients in need of long-term care, and mortality rate.

A “patient” or “subject” receiving the composition or treatment method of this invention is a human, including both adult and juvenile human, of any age, gender, and ethnic background, who has been diagnosed with COVID-19 (e.g., has had a positive nucleic acid and/or antibody test result for SARS-CoV2) and is in need of being treated to address PACS symptoms or to prevent the onset of such symptoms. Typically, the patient or subject receiving treatment according to the method of this invention to prevent or treat long COVID symptoms is not otherwise in need of treatment by the same therapeutic agents. For example, if a subject is receiving the symbiotic composition according to the claimed method, the subject is not suffering from any disease that is known to be treated by the same therapeutic agents. Although a patient may be of any age, in some cases the patient is at least 20, 30, 40, 45, 50, 55, 60, 65, 70, 75, 80, or 85 years of age; in some cases, a patient may be between 20 and 30, 30 and 40, 40 and 45 years old, or between 50 and 65 years of age, or between 65 and 85 years of age. A “child” subject is one under the age of 18 years, e.g., about 5-17, 9 or 10-17, or 12-17 years old, including an “infant,” who is younger than about 12 months old, e.g., younger than about 10, 8, 6, 4, or 2 months old, whereas an “adult” subject is one who is 18 years or older.

As used herein, the term “cohort” describes a group of subjects who are selected for a study based on one or more pre-determined features that are commonly shared among the subjects within the group.

The term “effective amount,” as used herein, refers to an amount that produces intended (e.g., therapeutic or prophylactic) effects for which a substance is administered. The effects include the prevention, correction, or inhibition of progression of the symptoms of a particular disease/condition and related complications to any detectable extent, e.g., incidence of disease, infection rate, one or more of the symptoms of a viral or bacterial infection and related disorder (e.g., COVID-19). The exact amount will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The term “about” when used in reference to a given value denotes a range encompassing ±10% of the value.

A “pharmaceutically acceptable” or “pharmacologically acceptable” excipient is a substance that is not biologically harmful or otherwise undesirable, i.e., the excipient may be administered to an individual along with a bioactive agent without causing any undesirable biological effects. Neither would the excipient interact in a deleterious manner with any of the components of the composition in which it is contained.

The term “excipient” refers to any essentially accessory substance that may be present in the finished dosage form of the composition of this invention. For example, the term “excipient” includes vehicles, binders, disintegrants, fillers (diluents), lubricants, glidants (flow enhancers), compression aids, colors, sweeteners, preservatives, suspending/dispersing agents, film formers/coatings, flavors and printing inks.

The term “consisting essentially of,” when used in the context of describing a composition containing an active ingredient or multiple active ingredients, refer to the fact that the composition does not contain other ingredients possessing any similar or relevant biological activity of the active ingredient(s) or capable of enhancing or suppressing the activity, whereas one or more inactive ingredients such as physiological or pharmaceutically acceptable excipients may be present in the composition. For example, a composition consisting essentially of active agents effective for treating COVID-19 or PACS in a subject is a composition that does not contain any other agents that may have any detectable positive or negative effect on the same target process (e.g., any one of the COVID-19 or PACS symptoms) or that may increase or decrease to any measurable extent of the relevant symptoms among the receiving subjects.

### DETAILED DESCRIPTION OF THE INVENTION

### I. Introduction

The present inventors have discovered based on analysis of multi-biome biomarkers and clinical factors that patients with COVID-19 can be classified into 2 clusters: the first cluster is associated with severe COVID-19 and PACS (multi-biome susceptible) and the second cluster is NOT associated with severe COVID-19 or PACS (multi-biome not susceptible). In this regard, this invention provides a novel method for assessing the risk of COVID-19 severity and risk of PACS by using a combination of multi-microbiome biomarkers and clinical markers as well as provides a method to reduce the risk of severe COVID-19 or PACS by modulating the gut microbiota.

The invention can be applied in an individual without COVID-19 to predict the risk of severe disease and PACS should they become infected with COVID-19. It can also be applied in a subject who had COVID-19 or have recovered from COVID-19 to predict their future risk of developing PACS. In subjects experiencing PACS or PACS-like symptoms regardless of whether COVID-19 has been diagnosed before, the method of this invention can help determine the likelihood of whether the symptoms are associated with COVID-19 based on their microbiome profile and clinical characteristics. In a second aspect, this invention provides a method to predict duration of SARS-CoV-2 virus shedding.

### II. Risk Assessment and Treatment

The present inventors discovered the use of multi-biome biomarker and clinical marker sets to determine microbiome susceptibility or association to severe COVID-19 and post-acute COVID syndrome (PACS, or long COVID) in a subject. Thus, the first step of the method of the present invention relates to assessing an individual's risk of developing severe COVID or long COVID, should the person become infected with SARS-CoV-2 by analyzing the pertinent multi-biome markers and clinical markers. Similar analysis may be performed to predict the likely time duration through which a person, in the event this person becomes infected with SARS-CoV-2, might continue to shed the virus and thus remain infectious.

Upon identifying any increased risk for severe or long COVID in a subject, the method of the present invention offers a further step of treating the person for the purpose of lowering such risk, for example, by modulating the level of certain microorganism species in the person's gastrointestinal tract, and therefore reducing the susceptibility to severe and/or long COVID or alleviating the relevant symptoms.

### A. Risk Assessment

In the first aspect, a person who may or may not have been diagnosed with COVID-19 and thus may or may not exhibit any COVID-related symptoms is assessed to ascertain whether he has severe COVID or long COVID, or to determine his risk of later developing severe COVID or long COVID. The person who is being tested is analyzed, the level or relative abundance of microorganism (bacterial, fungal, and viral species) set forth in Table 3 is determined in his stool sample, e.g., by PCR especially quantitative PCR. Also, the person is assessed in regard to those clinical parameters listed in Table 3. In the meantime, the level or relative abundance of these same microorganism species is determined by the same method as well as the same clinical parameters being assessed among the subjects of a reference cohort comprising COVID-19 patients, some of whom would eventually develop severe COVID or PACS whereas others would not develop severe COVID or PACS. Decision trees are then generated by random forest model using data obtained from the reference cohort, and the level or relative abundance of one or more of the microorganism species/the relevant clinical parameters from the individual being tested are run down the decision trees to generate a score indicative of risk for severe COVID or long COVID. The person is deemed to have severe COVID or PACS or have an increased risk for later developing severe COVID or PACS when his score is at least 0.5 (>0.5). In contrast, when his score is less than 0.5 (<0.5), the person is deemed to not suffer from severe COVID or PACS or have no increased risk for severe COVID or PACS.

In a second aspect, a person who has been diagnosed with COVID-19 is assessed for the purpose of predicting his virus shedding duration or the duration through which he will remain infectious. The person who is being tested is analyzed, the level or relative abundance of microorganism listed in Table 4 is determined in his stool sample, e.g., by PCR especially quantitative PCR. Also, the person is assessed in regard to those clinical parameters listed in Table 4. In the meantime, the level or relative abundance of these same microorganism species is determined by the same method as well as the same clinical parameters being assessed among the subjects of a reference cohort comprising COVID-19 patients, whose duration of viral shedding having been analyzed and determined. Decision trees are then generated by random forest model using data obtained from the reference cohort, and the level or relative abundance of one or more of the microorganism species/the relevant clinical parameters from the individual being tested are run down the decision trees to generate the viral shedding duration predicted for this particular COVID patient.

### B. Treatment Options

Once the severe COVID or long COVID risk assessment is made, for example, an individual who has been diagnosed as having an infection of SARS-CoV2 (e.g., based on a positive PCR or antibody/antigen test for SARS-CoV2) and who may not exhibit any of the clinical symptoms of the disease COVID-19 is deemed to have an increased risk of developing severe COVID or PACS at a later time, appropriate treatment steps can be taken as a measure to achieve the goal of preventing the onset of the severe COVID or PACS symptoms or reducing the number and/or severity of symptoms or eliminating the symptoms altogether. For instance, the patient may be given composition(s) comprising an effective amount of one or more of beneficial microbes such as Bifidobacterium adolescentis and Faecalibacterium prausnitzii, or the patient may be give composition(s) comprising an effective amount of one or more inhibitors specifically targeting for the suppression of detrimental microbes including bacteria Ruminococcus gnavus, Klebsiella species such as Klebsiella quasipneumonia, Klebsiella pneumoniae, and Klebsiella variicola, Clostridum species such as Clostridum bolteae and Clostridium innocuum and Clostridium spiroforme; fungi Asperigillus flavus, Candida glabrata, Candida albucans; and virus Mycobacterium phage MyraDee, Pseudomonas virus Pf1, and Klebsiella phage, e.g., by fecal microbiota transplantation (FMT) or by an alternative administration method via oral or local delivery, such that the microbiome in the patient's gastrointestinal tract will be modified to a profile that is favorable for the outcome of prevented, reduced, lessened, eliminated, or reversed severe COVID or PACS symptoms.

On the other hand, upon determining the duration of virus shedding in a COVID patient, care should be taken to ensure patient isolation or to keep the patient separate from the general population for at least the projected time period of viral shedding so as to eliminate or minimize the risk of disease transmission, while at the same time the patient may be administered therapeutic agents known in the pertinent field or disclosed here for COVID treatment.

### III. Pharmaceutical Compositions and Administration

The present invention provides pharmaceutical compositions comprising an effective amount of one or more of the beneficial bacterial species such as Bifidobacterium adolescentis and Faecalibacterium prausnitzii, or at least one specific inhibitor suppressing Ruminococcus gnavus, Klebsiella species (such as Klebsiella quasipneumonia, Klebsiella pneumoniae, and Klebsiella variicola), Clostridum species (such as Clostridum bolteae, Clostridium innocuum, and Clostridium spiroforme), Asperigillus flavus, Candida glabrata, Candida albucans, Mycobacterium phage MyraDee, Pseudomonas virus Pf1, and Klebsiella phage, or any combination thereof, which are useful for treating a COVID-19 patient to reduce the risk of developing symptom(s) of severe COVID or PACS or to ameliorate the symptom(s) if any already present. Pharmaceutical compositions of the invention are suitable for use in a variety of drug delivery systems. Suitable formulations for use in the present invention are found in Remington's Pharmaceutical Sciences, Mack Publishing Company, Philadelphia, PA, 17th ed. (1985). For a brief review of methods for drug delivery, see, Langer, Science 249: 1527-1533 (1990).

The pharmaceutical compositions of the present invention can be administered by various routes, e.g., systemic administration via oral ingestion or local delivery using a rectal suppository. The preferred route of administering the pharmaceutical compositions is oral administration at suitable daily doses. When multiple bacterial species and/or inhibitors (e.g., antisense oligonucleotides, small interfering/inhibitory RNA such as miRNA, siRNA, and dsRNA etc.) specifically targeting one or more particular species selected from Ruminococcus gnavus, Klebsiella species (such as Klebsiella quasipneumonia, Klebsiella pneumoniae, and Klebsiella variicola), Clostridum species (such as Clostridum bolteae, Clostridium innocuum, and Clostridium spiroforme), Asperigillus flavus, Candida glabrata, Candida albucans, Mycobacterium phage MyraDee, Pseudomonas virus Pf1, and Klebsiella phage) are administered to the subject, they may be administered either in one single composition or in multiple compositions. The appropriate dose may be administered in a single daily dose or as divided doses presented at appropriate intervals, for example as two, three, four, or more subdoses per day. The duration of administration may range from about 1 week to about 8 weeks, e.g., about 2 week to about 4 weeks, or for a longer time period (e.g., up to 6 months) as the relevant symptoms persist.

For preparing pharmaceutical compositions containing the beneficial bacteria identified in this disclosure, one or more inert and pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., any one or more of the beneficial bacterial species Bifidobacterium adolescentis and Faecalibacterium prausnitzii. In tablets, the active ingredient is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 100% by weight of the active ingredient(s) (e.g., one or more of the beneficial bacterial species named above or one or more inhibitors specifically targeting the detrimental microbial species named above and herein). Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active ingredient(s), e.g., one or more of the beneficial bacterial species named above or one or more inhibitors specifically targeting the detrimental microbial species named above and herein, with encapsulating material as a carrier providing a capsule in which the active ingredient(s) (with or without other carriers) is surrounded by the carrier, such that the carrier is thus in association with the active ingredient(s). In a similar manner, sachets can also be included. Tablets, powders, sachets, and capsules can be used as solid dosage forms suitable for oral administration.

Liquid pharmaceutical compositions include, for example, solutions suitable for oral administration or local delivery, suspensions, and emulsions suitable for oral administration. Water-based solutions made from adding into previously sterilized aqueous solutions the active component(s) (e.g., one or more of the beneficial bacterial species named above or one or more inhibitors specifically targeting the detrimental microbial species named above and herein) in solvents comprising water, buffered water, saline, PBS, ethanol, or propylene glycol are examples of liquid or semi-liquid compositions suitable for oral administration or local delivery such as by rectal suppository. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., one or more of inhibitors specifically targeting the detrimental microbial species named above and herein) in the desired solvent system, and then passing the resulting solution through a membrane filter to sterilize it or, alternatively, by dissolving the sterile active component in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

Single or multiple administrations of the compositions can be carried out with dose levels and pattern being selected by the treating physician. In any event, the pharmaceutical formulations should provide a quantity of an active agent sufficient to effectively enhance the efficacy of a vaccine and/or reduce or eliminate undesirable adverse effects of a vaccine.

### IV. Additional Therapeutic Agents

Additional known therapeutic agent or agents may be used in combination with an active agent, such as one or more of the beneficial bacterial species such as Bifidobacterium adolescentis and Faecalibacterium prausnitzii, or at least one specific inhibitor suppressing Ruminococcus gnavus, Klebsiella species (such as Klebsiella quasipneumonia, Klebsiella pneumoniae, and Klebsiella variicola), Clostridum species (such as Clostridum bolteae, Clostridium innocuum, and Clostridium spiroforme), Asperigillus flavus, Candida glabrata, Candida albucans, Mycobacterium phage MyraDee, Pseudomonas virus Pf1, and Klebsiella phage, or any combination thereof, in the practice of the present invention for the purpose of treating or preventing severe COVID or long COVID symptom(s) in a patient or for the purpose of reducing viral shedding duration in a patient. In such applications, one or more of the previously known effective prophylactic/therapeutic agents can be administered to patients concurrently with an effective amount of the active agent(s) either together in a single composition or separately in two or more different compositions.

For example, drugs and supplements that are known to be effective for use to prevent or treat COVID-19 include ivermectin, vitamin C, vitamin D, melatonin, quercetin, Zinc, hydroxychloroquine, fluvoxamine/fluoxetine, proxalutamide, doxycycline, and azithromycin. They may be used in combination with the active agents (such as any one or more of the beneficial bacterial species named herein and/or any one or more specific inhibitors of the detrimental microbial species named herein) of the present invention to promote safe and full recovery among patients suffering from SARS-CoV2 infection, reduce potential disease severity (including morbidity and mortality), limiting the time duration of active viral shedding, and ensure elimination of any serious or lingering long-term ill effects from the disease. In particular, the combination of Zinc, hydroxychloroquine, and azithromycin and the combination of ivermectin, fluvoxamine or fluoxetine, proxalutamide, doxycycline, vitamin C, vitamin D, melatonin, quercetin, and Zinc have demonstrated high efficacy in both COVID prophylaxis and therapy. Thus, these known drug/supplement or nutritheutical combinations can be used in the method of this invention along with the active components of one or more of the beneficial bacterial species named herein and/or one or more of the specific inhibitors suppressing the detrimental microbial species named herein.

### V. Kits

The invention also provides kits for treating and preventing severe and/or long COVID symptoms among patients as well as for reducing the duration of active virus shedding in COVID patients in accordance with the methods disclosed herein. The kits typically include a plurality of containers, each containing a composition comprising one or more of the beneficial bacterial species such as Bifidobacterium adolescentis and Faecalibacterium prausnitzii, or at least one specific inhibitor suppressing Ruminococcus gnavus. Klebsiella species (such as Klebsiella quasipneumonia, Klebsiella pneumoniae, and Klebsiella variicola), Clostridum species (such as Clostridum bolteae, Clostridium innocuum, and Clostridium spiroforme), Asperigillus flavus, Candida glabrata, Candida albucans, Mycobacterium phage MyraDee, Pseudomonas virus Pf1, and Klebsiella phage, or any combination of the above. Further, additional agents or drugs that are known to be therapeutically effective for prevention and/or treatment of the disease, including for ameliorating the symptoms and reducing the severity of the disease, as well as for facilitating recovery from the disease (such as those described in the last section or otherwise known in the pertinent technical field) may be included in the kit. The plurality of containers of the kit each may contain a different active agent/drug or a distinct combination of two or more of the active agents or drugs. The kit may further include informational material providing instructions on how to dispense the pharmaceutical composition(s), including description of the type of patients who may be treated (e.g., human patients, adults or children, who have been diagnosed of COVID-19 and deemed to suffer from or to be at risk of later developing severe COVID or PACS), the dosage, frequency, and manner of administration, and the like.

### EXAMPLES

The following examples are provided by way of illustration only and not by way of limitation. Those of skill in the art will readily recognize a variety of non-critical parameters that could be changed or modified to yield essentially the same or similar results.

### Background

The coronavirus disease-2019 (COVID-19) pandemic has affected over 450 million people and killed 6 million people worldwide. Identifying predictors of disease severity and deterioration is a priority to guide clinicians and policymakers for better clinical management, resource allocation and long-term management of COVID-19 patients. Several lines of evidence such as replication of severe acute respiratory syndrome coronavirus 2 (SARS-CoV-2) in human enterocytes¹⁻³, detection of viruses in fecal samples^{4,5} and altered gut microbiota composition including increased abundance of opportunistic pathogens and reduced abundance of beneficial symbionts in the gut of patients with COVID-19 suggest involvement of the gastrointestinal (GI) tract⁶⁻⁹.

Recent studies showed that gut dysbiosis is linked to severity of COVID-19 infection and persisted complications months after disease resolution^{7,8,10}. Patients with severe disease had elevated plasma concentrations of inflammatory cytokines and markers including interleukin-6 (IL-6), IL-8 and IL-10 and lactate dehydrogenase (LDH) and C reactive protein (CRP) reflecting immune response and tissue damage from SARS-CoV-2 infection^{11,12}. Among hospitalized patients with COVID-19, gut microbiota composition was also associated with blood inflammatory markers⁷, and lack of short chain fatty acids and L-isoleucine biosynthesis in the gut microbiome correlated with disease severity¹³.

Beyond bacteria, the human gut is also home to a vast number of viral and fungi communities which regulate host homeostasis, physiological processes and the assembly of co-residing gut bacteria, which could potentially play an important role in the pathophysiological mechanisms determining COVID-19 outcomes. Since therapeutic potentials for COVID-19 patients include approaches to inhibit, activate, or modulate immune function, it is essential to define characteristics related to clinical features in a well-defined patient cohort. We hypothesized that microbial interaction networks may provide improved understanding of the pathophysiology and long-term consequences of COVID-19. Here, using an unsupervised classification approach based on fecal metagenomic profiling and blood inflammatory markers, we demonstrated that integrative microbiomes from a multi-kingdom network provide a novel framework for understanding disease complications and has potential applications for risk stratification and prognostification of COVID-19. This invention relates to the use of a combination of multi-microbiome biomarkers and clinical markers (from stool and blood samples) to determine susceptibility to, or, association with severe COVID-19 and PACS in a subject. The invention further comprises steps to lower the risk by modulating the gut microbiota. In a second aspect, this invention provides a method to predict duration of SARS-CoV-2 virus shedding.

### Multi-Omics Analysis Reflects Disease Severity and Clinical Symptoms in COVID-19

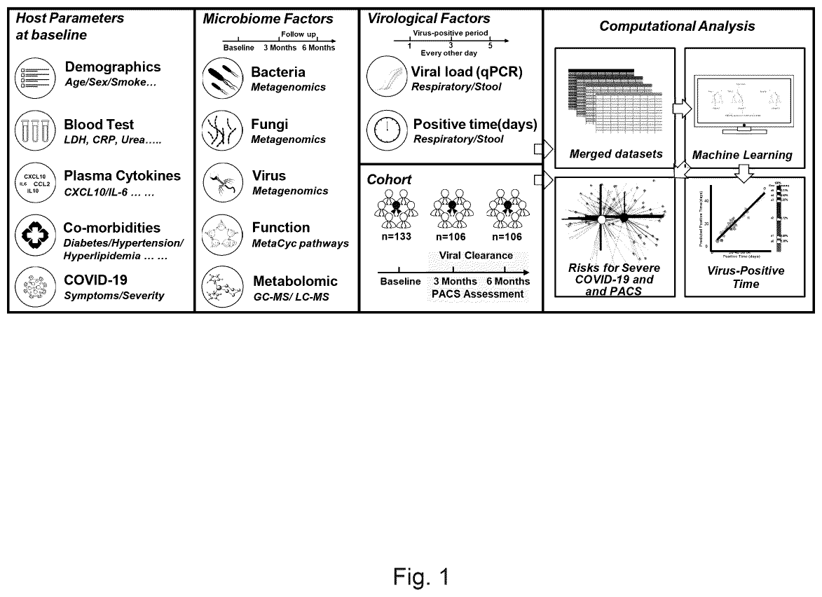

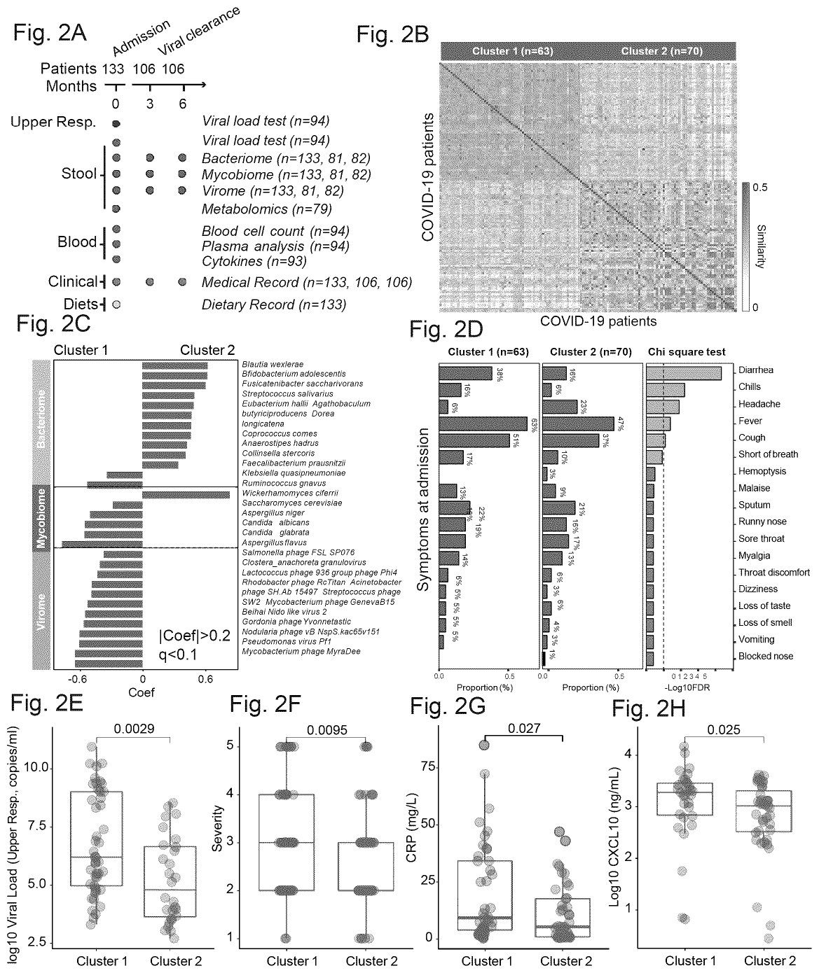

We included 133 hospitalised patients with COVID-19 in three hospitals in Hong Kong from between 13 Mar. 2020 and 27 Jan. 2021. We assessed viral RNA load quantified by quantitative PCR with reverse transcription (RT-qPCR) using nasopharyngeal swabs and fecal samples, plasma cytokines and chemokines levels and leukocyte profiles by flow cytometry using freshly isolated peripheral blood mononuclear cells (PBMCs). We also analysed microbiome (bacteria, virus, fungi) composition of 293 serial faecal samples with three longitudinal time-points from admission to six months after virus clearance using shotgun metagenomic sequencing and assessed metabolomics of 79 faecal samples at admission (FIG. 1, FIG. 2A).

Baseline gut multi-biome (bacteria, fungi, virus) profile was integrated by an unsupervised weighted similarity network fusion (WSNF) approach. Weighting was assigned according to the total number of observed taxa present in a particular biome, with filtering based on a prevalence of at least 5% across the patient cohort; that was, virome (732 species)>bacteriome (242 species)>mycobiome (12 species) observed across 133 patients. By pooling and subjecting multi-biome data to a non-supervised similarity network fusion approach, faecal samples were divided two distinct patient clusters based on microbiota matrix: 47.36% of patients in WSF-Cluster 1 (n=63), and 52.63% in WSF-Cluster 2 (n=70, FIG. 2B).

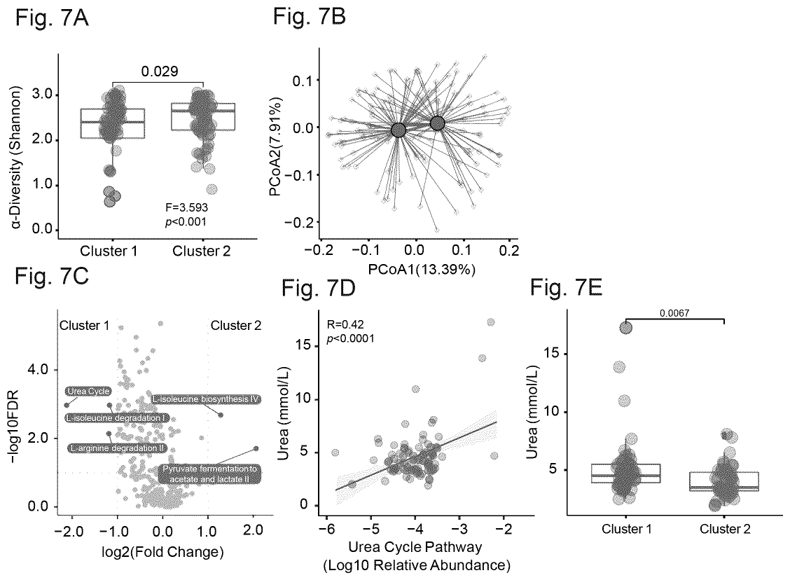

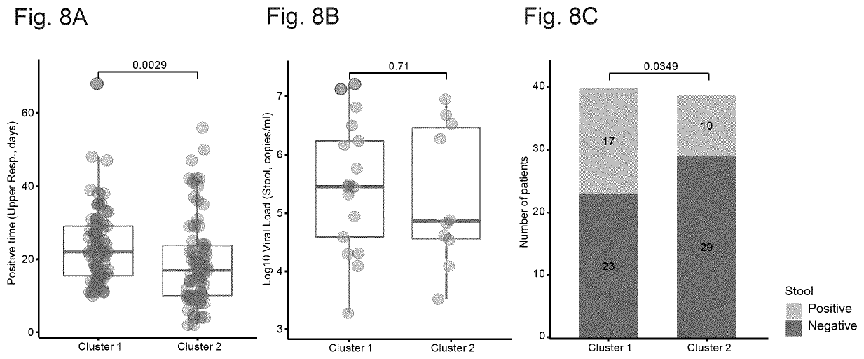

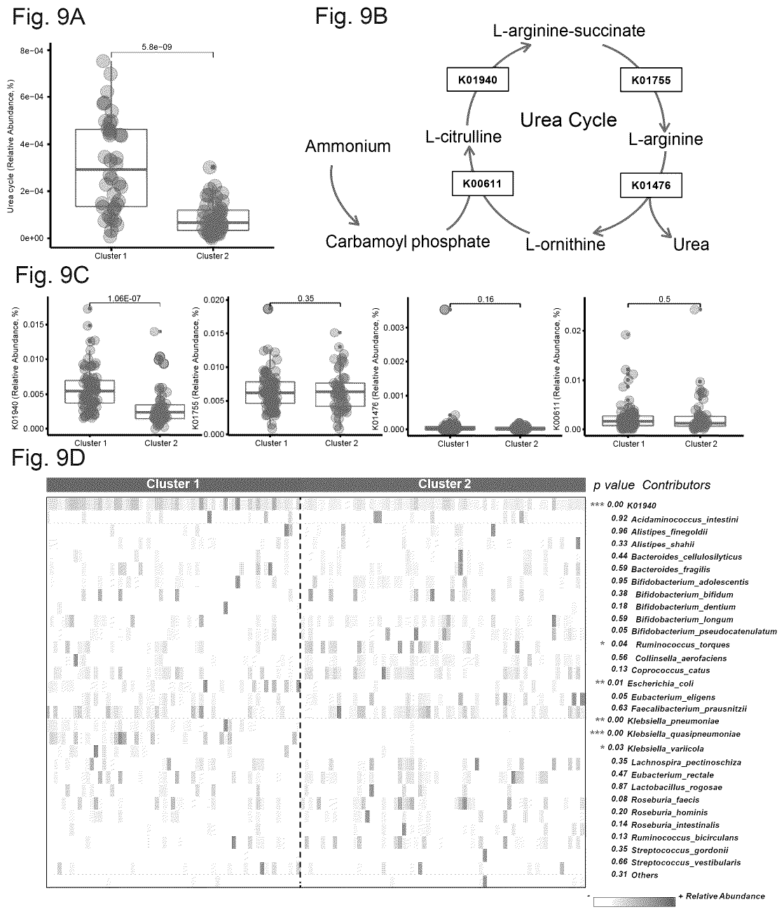

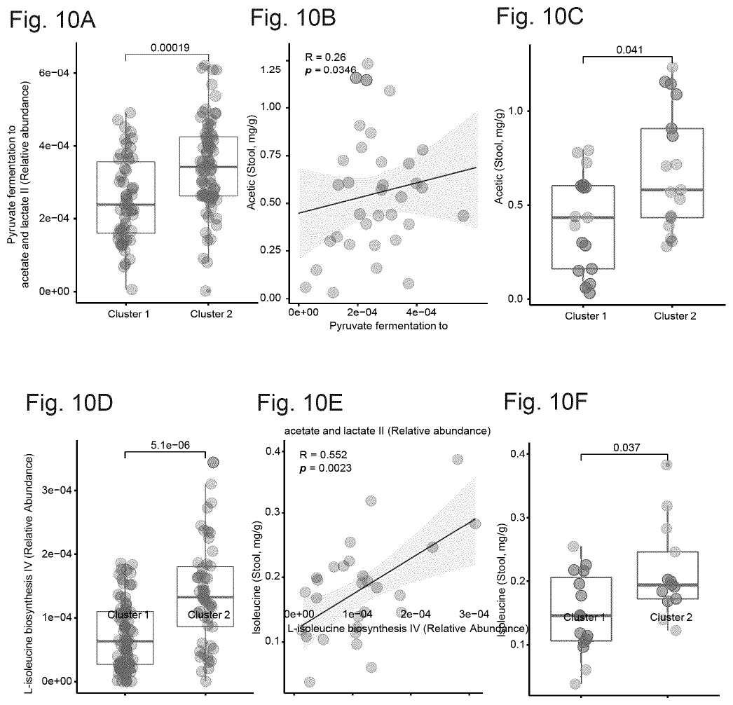

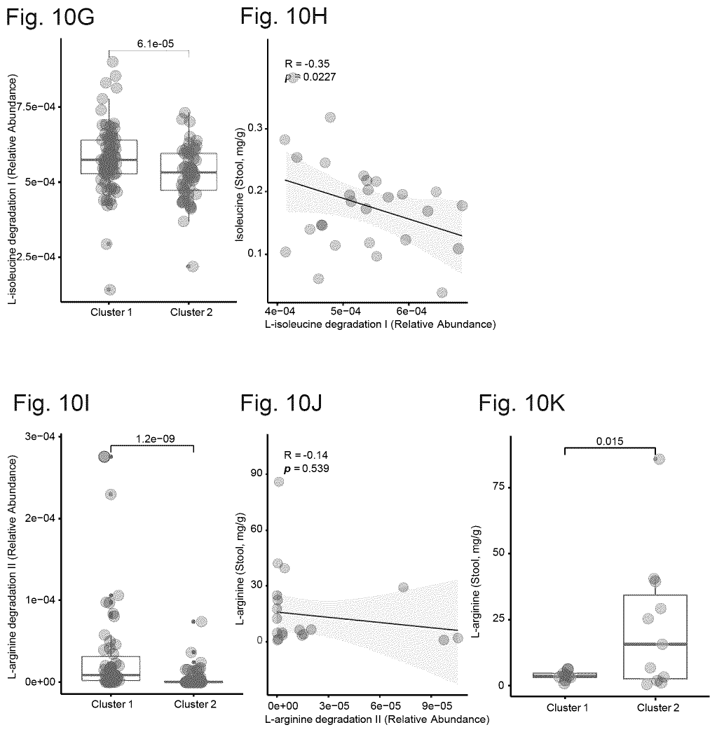

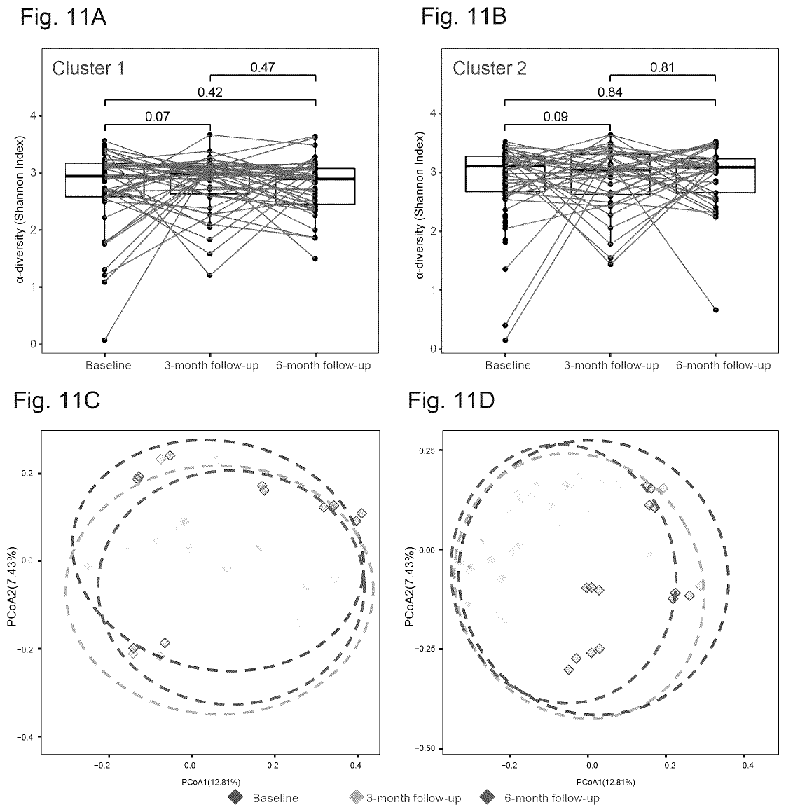

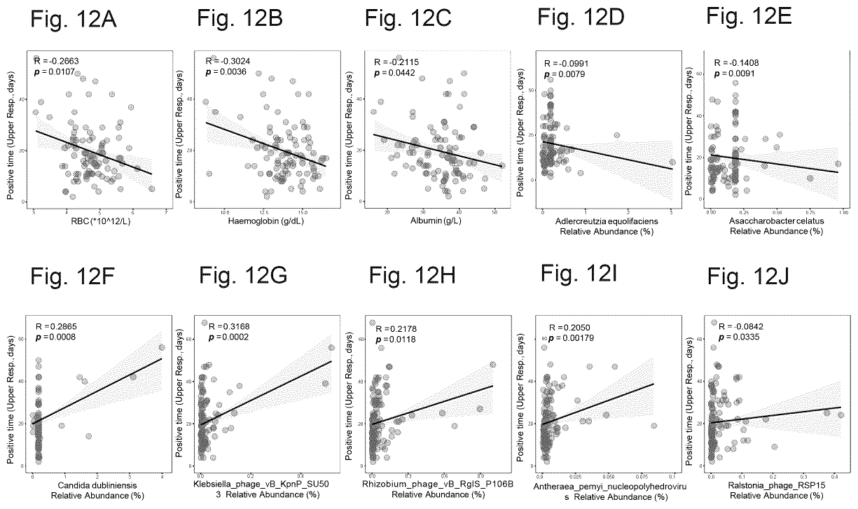

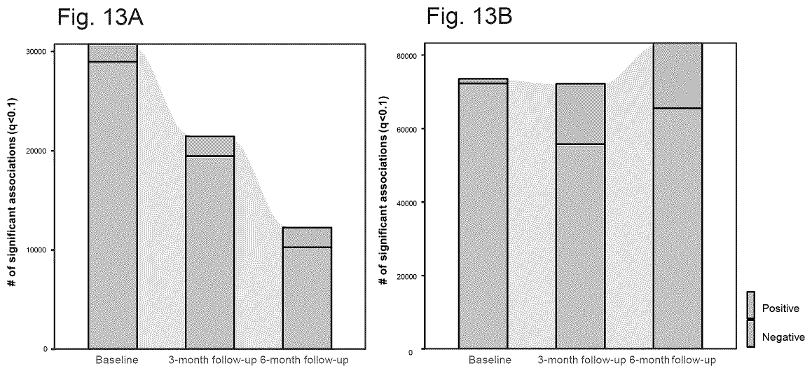

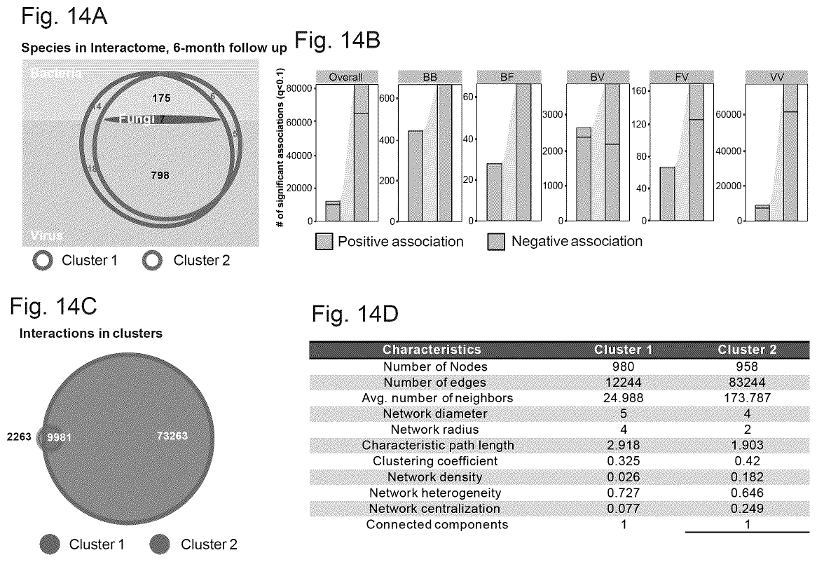

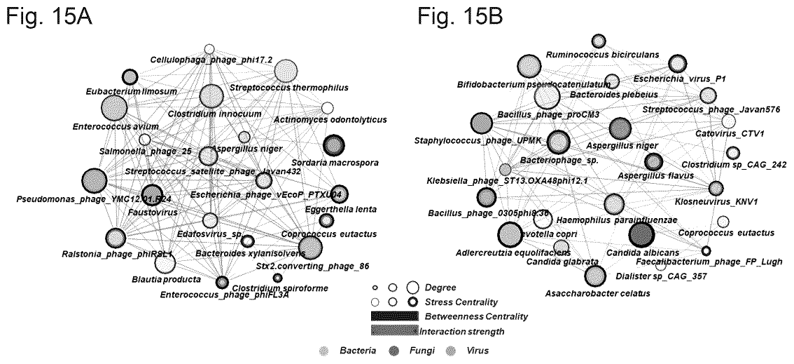

We next compared microbial profiles between clusters (adjusted for age, sex and comorbidity). Multi-biome composition of patients in Cluster 1 was characterized by a predominance of bacteria Ruminococcus gnavus, Klebsiella quasipneumoniae, fungi Asperigillus flavus, Candida glabrata, Candida albucans and virus Mycobacterium phage MyraDee. Pseudomonas virus Pf1 (FIG. 2C, MaAslin 2, q<0.1, Supplementary Table 1). They also exhibited significantly lower multi-biome diversity (Mann-Whitney test, p=0.029, FIG. 7A) than those in Cluster 2. Principal Coordinates Analysis (PCoA) of multi-biome composition showed significant difference between the two clusters using permutational multivariate analysis of variance (PERMANOVA) analysis (p<0.001, FIG. 7B).

We found that patients belonging to Cluster 1 exhibited more symptoms such as diarrhoea and chills (2-fold increase risk) and fever and cough (1.3-fold increase risk; Chi-square, p value<0.001, q<0.1) than those in Cluster 2 at admission (FIG. 2D). They were also characterized by a higher viral load (FIG. 2E), worse disease severity (FIG. 2F), increased CRP (FIG. 2G), increased CXCL10 (FIG. 2H), longer duration of viral positivity from upper respiratory tract samples (FIG. 8A) and higher rate of viral positivity in faecal samples (FIG. 8B) than those in Cluster 2. Demographics and comorbidities were comparable between Cluster 1 and Cluster 2, except patients within Cluster 1 were 9.2 years older than those in Cluster 2 (Table 1). Patients in Cluster 1 primarily comprised subjects with severe COVID-19 who had more clinical signs (FIG. 2F, FIG. 2D) and these subjects presented with higher plasma CRP and chemokines levels such as CXCL10 known to be involved in leukocyte trafficking^{14,15}. These observations indicate that gut multi-biome profile of COVID-19 patients at admission are associated with disease severity, and Cluster 1 was defined by more severe disease.

TABLE 1
Comparison of clinical characteristics in COVID-19 patients
stratified by integrative multi-kingdom microbiome

| | Overall | | Cluster 1 | | Cluster 2 | | p |
|---|---|---|---|---|---|---|---|
| Patients, n | 133 | | 63 | | 70 | | |
| Female, n (%) | 59 | (44.4%) | 30 | (47.6%) | 29 | (41.4%) | 0.207 |
| Age, years (IQR) | 42.2 | (26-59) | 47.1 | (28.5-63) | 37.9 | (20.5-55) | 0.005 |
| Non-smokers, n (%) | 72 | (54.1%) | 34 | (53.9%) | 38 | (54.3%) | 0.402 |
| Presence of any comorbidities, n (%) | 52 | (39.1%) | 25 | (39.7%) | 27 | (38.6%) | 0.393 |
| Hypertension | 28 | (21.1%) | 11 | (17.5%) | 18 | (25.7%) | 0.099 |
| Hyperlipidaemia | 25 | (18.8%) | 11 | (17.5%) | 14 | (20.0%) | 0.335 |
| Diabetes mellitus | 10 | (7.5%) | 4 | (6.3%) | 6 | (8.6%) | 0.287 |
| Length of stay in hospital, days (IQR) | 21.4 | (13-28) | 23.8 | (15.5-29) | 19.22 | (10-23.75) | 0.025 |
| Severity of COVID-19, n (%) | | | | | | | 0.010 |
| Asymptomatic | 7 | (5.3%) | 2 | (3.2%) | 5 | (7.1%) | 0.153 |
| Mild | 52 | (39.1%) | 19 | (30.2%) | 33 | (47.1%) | 0.007 |
| Moderate | 47 | (35.3%) | 25 | (39.7%) | 22 | (31.4%) | 0.114 |
| Severe | 15 | (11.3%) | 9 | (14.3%) | 6 | (8.6%) | 0.080 |
| Critical | 12 | (9.0%) | 8 | (12.7%) | 4 | (5.7%) | 0.016 |

We explored functional profiling of microbiome signatures in the two clusters and identified cluster-specific functional signatures (FIG. 7C, Supplementary Table 2). Amongst all microbiome functionalities, urea cycle, L-isoleucine degradation I and L-arginine degradation II function were enriched in Cluster 1 (FIG. 7C, q<0.1, fold change>2). Elevated blood urea nitrogen (BUN) level has been reported to be associated with critical illness and mortality in patients with COVID-19 and was predictive of poor clinical outcome^{14,16}. We found that blood urea levels were strongly associated with microbiome urea cycle pathway and showed higher concentrations in COVID-19 patients with severe disease (FIG. 7D, FIG. 7E, FIG. 9A). Next, we investigated whether specific microbiome species were associated with elevated urea in severe COVID-19. By comparing the subclasses pathway and microbial contributors (quantify gene presence and abundance in a species-stratified manner), we found a marked increase in K01940 (argininosuccinate synthase, the key enzyme in urea cycle pathway, FIG. 9B) in the severe cluster (FIG. 9C), which was predominantly driven by Klebsiella species such as Klebsiella quasipneumonia, Klebsiella pneumoniae and Klebsiella variicola (FIG. 9D). High urea level is commonly an indication of kidney dysfunction. However, in our cohort, there was no significant difference in other blood markers of liver and kidney functions (total protein, ALP, ALT, creatinine, Supplementary Table 3, Supplementary Table 4) except blood urea. Given signatures that correlated with disease deterioration, gut-derived uremic toxins into the systemic circulation might be one of the explanations for the marked increase of urea in severe COVID-19 patients. Enriched L-isoleucine degradation I and L-arginine degradation II, and decreased L-isoleucine biosynthesis IV, as well as pyruvate fermentation to acetate and lactate II, were further verified by metabolomics sequencing and correlation analysis (FIG. 10).

### Integrative Microbiome Signatures and Post-Acute COVID-19 Syndrome (PACS)

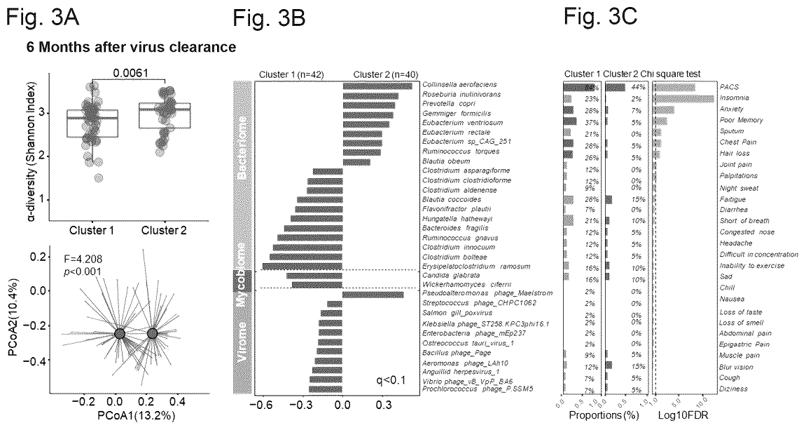

An exaggerated immune system response, cell damage, or the physiological consequences of COVID-19 may contribute to persistent and prolonged effects after acute COVID-19 known as post-acute COVID-19 syndrome (PACS). The exact pathophysiological mechanism underlying PACS is unclear^{10,17,18}. By following gut microbiome dynamics of patients with COVID-19 from admission until six months after virus clearance, we explored baseline microbiome composition (bacteria, virus, fungi) and association with development of PACS. Within Cluster 1 and Cluster 2, there was no significant difference in the gut microbiome composition at baseline and follow-up samples (FIG. 11A-5D) within each cluster suggesting the gut microbiome profile were stable over time. At six months, patients in Cluster 1 had significantly different gut microbiota composition than those in Cluster 2 (FIG. 3A). Bacteria diversity in Cluster 1 was significantly lower than that of Cluster 2 (FIG. 3A, p=0.0061). Cluster 1 was characterised by increased in pathogenic bacteria species including Clostridum bolteae and Clostridium innocuum at six months (adjusted for age, sex and comorbidity, FIG. 3B). Significantly more patients within Cluster 1 (84% vs 44%; FDR<0.1, Chi square test) developed symptoms of PACS including insomnia (23% vs 2%; FDR<0.1), anxiety (28% vs 7%; FDR<0.1) and poor memory (37% vs 5%; FDR<0.1) compared with those in Cluster 2 (FIG. 3C).

### Incorporation of Host Factors Improves the Performance of Classification Model

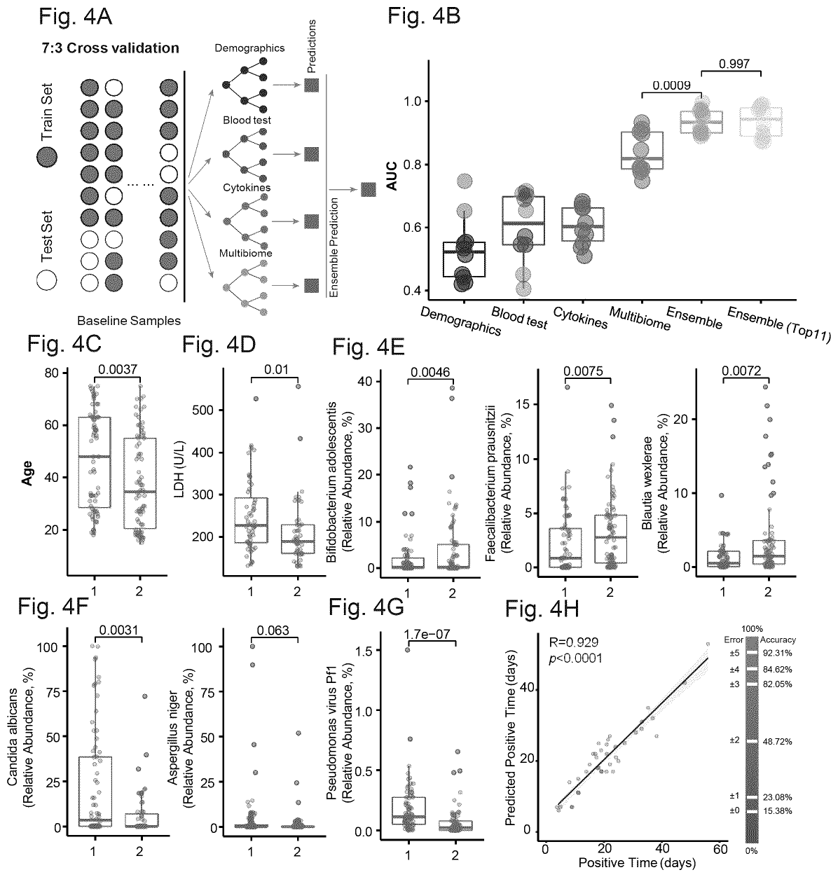

We next incorporated host parameters (patient demographics, blood parameters, cytokine levels) with microbiome analysis. Using random forest modelling of both host factors and microbiome signatures and a stratified ten-fold cross-validation (FIG. 4A), this model can differentiate Cluster 1 and Cluster 2 with an area-under receiving operator curve (AORUC) of 0.94 (FIG. 4B, Supplementary Table 5). In contrast, a model that incorporated patient demographics (i.e. age, gender, co-morbidities), blood parameters (CRP, LDH), cytokines (i.e. CXCL10, IL 1b, IL 10), and microbiome analysis alone achieved an AUC of 0.53, 0.60, 0.61 and 0.84, respectively in differentiating the two clusters (Supplementary Table 5). Patients in Cluster 1 were characterised by more advanced age, higher LDH level, higher relative abundance of Candida albicans and Pseudomonas phages Pf1 and lower relative abundance of Bifidobacterium adolescentis and Faecalibacterium prausnitzii (FIG. 4C-4G). Further limitation to the top eleven factors on random forest, our model achieved an AUC of 0.98 to differentiate between the two clusters. These eleven factors included host factors (age, viral load, blood LDH, CRP and CXCL10 levels), bacteria (Bifidobacterium adolescentis, Faecalibacterium prausnitzii, Blautia wexlerae), fungi (Candida albicans, Aspergillus niger) and virus (Pseudomonas virus Pf1) composition (Table 2, Supplementary Table 6). These data indicate that host and microbial factors in combination provide the most accurate discriminating ability in defining subjects with severe COVID-19.

### Machine Learning Model for COVID-19 Prognosis Including Prediction of COVID-19 Severity and PACS

The gut microbiome profile was stable over time as the gut microbiome composition at baseline samples showed no significant different from that of follow-up samples within Cluster 1 (multi-biome susceptible or associated) and Cluster 2 (multi-biome not susceptible). Therefore, this model can be applied to a subject at any time, including prior to COVID-19 infection, at the time of COVID-19 symptom onset or diagnosis, or after recovery from COVID-19 infection. This model can also be applied to a subject who is experiencing PACS-like symptoms without a prior positive test of COVID-19. This model does not target the virus itself, making it suitable for all COVID-19 variants. Subjects classified as “multi-biome susceptible or associated” using this model are deemed to be at higher risk of presenting with symptoms, blood and viral parameters and clinical outcome, or with PACS as listed in Table 2.

TABLE 2
List of symptoms or clinical markers that are more
likely to be present in subjects within cluster
1 (multi-biome susceptible or associated)

During COVID-19 infection
Diarrhoea
Chills
Fever
Cough
Higher Viral Load
Worse disease severity
Increased CRP
Increased CXCL10
Longer viral shedding
Higher rate of viral positivity in fecal samples
PACS
Insomnia
Poor memory

TABLE 3
Factors included in the Prediction of Multi-biome Susceptibility
or Association with Severe COVID-19 or PACS

| | | Mean Value/Mean | |
|---|---|---|---|
| | NCBI:txid | Relative abundance | |
| Factors | (if applicable) | Cluster 1 | Cluster 2 |
| Age | NA | 47.1 | 37.9 |
| Viral load | NA | 6.76 | 4.89 |
| LDH | NA | 249.2 | 210.6 |
| CRP | NA | 19.9 | 11.1 |
| CXCL10 | NA | 2583.2 | 1326.3 |
| Bifidobacterium | 1680 | 1.86 | 3.97 |
| adolescentis | | | |
| Faecalibacterium | 853 | 2.13 | 3.37 |
| prausnitzii | | | |
| Blautia wexlerae | 418240 | 1.44 | 3.80 |
| Candida albicans | 5476 | 4.76 | 21.80 |
| Aspergillus niger | 5061 | 1.66 | 6.62 |
| Pseudomonas virus Pf1 | 2011081 | 0.00191 | 0.000801 |

To determine whether a subject is susceptible to or have severe COVID-19 or PACS, the following steps are carried out:
(1) Obtain a set of training data by determining the relative abundance of species and other clinical factors selected from Table 3 in a cohort of COVID-19 patients with and without severe COVID-19, PACS or symptoms listed in Table 2.
(2) Determine the relative abundance of these species and other clinical factors selected from Table 3 in the subject who is being tested for multi-biome susceptibility or multi-biome-associated with serious COVID-19 or PACS.
(3) Compare the relative abundance of these species and other clinical factors selected from Table 3 in the subject with the training data using random forest model.
(4) Decision trees will be generated by random forest from the training data. The relative abundances and values of other clinical factors will be run down the decision trees and generate a risk score. If at least 50% trees (possibility ≥0.5) in the model then consider the subject as COVID-19 patients having susceptible multi-biome or multi-biome-associated with severe COVID-19 or PACS, hence the subject being tested is deemed to have an increased risk for severe COVID-19 or PACS. If less than 50% trees (possibility <0.5) in the model consider the subject as not having susceptible multi-biome or multi-biome-associated with severe COVID-19 or PACS, the subject being tested is deemed to not have an increased risk for serious COVID-19 or PACS.

### Machine Learning Model for the Prediction of Viral Shedding Duration and for the Determination of Isolation Duration Following COVID-19 Infection

To explore whether integration of clinical data with deep microbiome profiling could predict the duration of viral shedding in COVID-19, we tested 1,378 samples from the upper respiratory tract (sputum and nasopharyngeal samples) for the presence of SARS-CoV-2 virus RT-qPCR every two days for each patient. The median duration of viral shedding (based on positive RT qPCR) was 21.1 days (IQR 14.5-24.5, range 4-56) after onset of initial symptoms. We used random forest analysis of ensembled datasets (demographic, blood test, cytokines and multi-biome) to predict the duration of viral shedding in an individual patient Using a discovery cohort of 93 patients with COVID-19 followed by a test cohort of 40 patients, our predictive model produced an accuracy of 82.06% with error 3 days for predicting duration of viral shedding (FIG. 4H). The taxa that contributed most to the model were fungi and bacteriophages including Candida dubliniensis, Klebsiella_phage_vB_KpnP_SU50, and Rhizobium_phage_vB_RglS_P106B (FIG. 12), and these markers could be considered in determining length of viral shedding. Since viral shedding is associated with disease transmission, this model can be used to guide the discontinuation or de-escalation of infection prevention and control precautions.

TABLE 4
Factors included in the Machine Learning Model for Prediction
of Viral Shedding Duration and the Determination of
Isolation Duration following COVID-19 Infection

| Factors | NCBI:txid (if applicable) |
|---|---|
| RBC | NA |
| Haemoglobin | NA |
| Albumin | NA |
| Adlercreutzia equolifaciens | 446660 |
| Asaccharobacter celatus | 394340 |
| Candida dubliniensis | 42374 |
| Klebsiella_phage_vB_KpnP_SU503 | 1610834 |
| Rhizobium_phage_vB_RgIS_P106B | 1458697 |
| Antheraea_pernyi_nucleopolyhedrovirus | 161494 |
| Ralstonia_phage_RSP15 | 1785960 |

To determine the viral shedding duration and/or isolation duration in a subject following COVID-19 infection, the following steps are carried out:
(1) Obtain a set of training data by determining the relative abundance of species and other clinical factors selected from Table 4 in a cohort of COVID-19 patients and their SARS-CoV-2 viral shedding duration (Upper respiratory tract). The random forest model was used to regress features from ensembled data set (demographic, blood test, cytokines and multibiome) in the time-series profiling of COVID-19 patients against their SARS-CoV-2019 positive time (Upper respiratory tract) using default parameters of R package randomForest v4.6-14.
(2) Determine the relative abundance of these species and other clinical factors selected from Table 4 in the subject who is being tested to predict the duration of viral shedding
(3) Compare the relative abundance of these species and values of other clinical factors in the subject with the training data using random forest model.
(4) Forecasted viral shedding duration will be generated by the random forest model.

### Network Analysis of the Interactome of COVID-19 Patients

We performed network analysis of the interactions of bacteriome, mycobiome and virome to investigate the co-occurrence of multi-biome signatures in patients from the two clusters: Cluster 1 (severe) and Cluster 2 (non-severe). We first conducted co-occurrence analysis by an ensemble of similarity and regression approaches to generate association networks. Taxa with close evolutionary relationships tended to positively correlate while distantly related microorganisms with functional similarities tended to compete¹⁹. Herein, a positive interaction of microorganisms was defined by a correlative score representing the co-occurrence of microbes while a negative value indicates co-exclusion. We found that patients in the non-severe cluster had a higher total number of bacteria whereas a lower number of viruses in the multi-interactome (FIG. 5A). Intriguingly, we found reduced number of negative associations among bacteria-viruses and fungi-viruses in microbiome of severe cluster (FIG. 5B), indicating decreased co-exclusion trans-kingdom patterns in patients with severe disease. The lack of interactions of bacteria-viruses and fungi-viruses in patients in Cluster 1 included invasive gut pathogen Ruminococcus gnavus, Clostridium spiroforme and two fungi hubs of Candida albicans and Wickerhamomyces ciferrii (FIG. 5C, FIG. 5D). Furthermore, the number of interactions in the non-severe cluster was three times higher than that of the severe cluster (FIG. 5E). Taking Clostridium spiroforme as an example, it was mainly positively correlated with other constituent microbes in the severe cluster but negatively correlated in the non-severe cluster (FIG. 5F). These findings highlight a preferential mechanism on the loss of inhibitory effect of pathogenic microbes in the severe group.

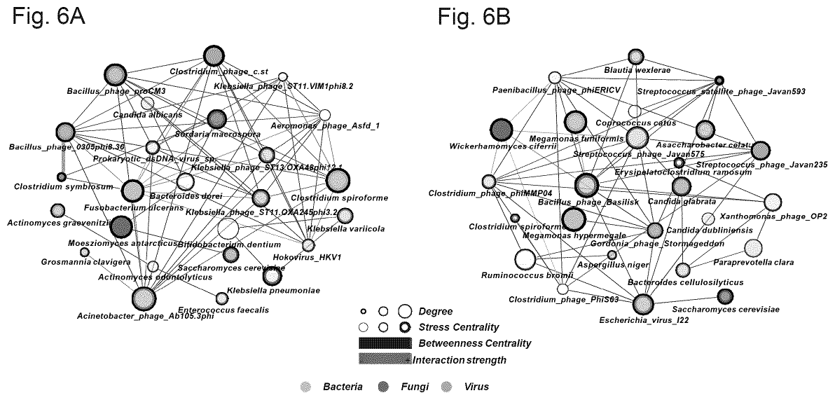

We next examined the network metrics node degree, stress centrality, betweenness centrality (of the nodes) to depict impact of microbes on the network integrity. In the severe cluster, Klebsiella pneumoniae, Clostridium spiroforme, and Klebsiella phage represented the highest-ranked taxa (FIG. 6A, Supplementary table 7, Supplementary table 8). The top representative taxa were not commonly shared in the non-severe cluster. The important characteristic of the network for the non-severe cluster was explained by the interaction of microbes such as Ruminococcus bromii and Saccharomyces cerevisiae with other taxonomic members (FIG. 6B). This observation indicates that the interactome of a microbe, rather than the microbe itself, dictates clinical status such as the severity of COVID-19. To further assess microbial interactions in relation to development of PACS, the network metrics were compared. From admission to 6-months, interactomes of microbiome in the severe cluster group was further reduced while that of the non-severe cluster changed moderately with more negative correlations (FIG. 13A, 7B). Cluster 1 was persistently characterised by fewer negative interactions between microbes suggesting a loss of negative microbial interactions that potentially drives clinical status of PACS (FIG. 14). At six months, each interaction network contained a range of discriminant bacterial, fungal and viral taxa: the core network in the severe cluster included Streptococcus thermophilus, Sordaria macrospora, Stx2.converting_phage_86 (FIG. 15).

### Discussion

Our cross-sectional and prospective multi-omics analysis revealed several new insights of the role of host and microbial factors in COVID-19 severity and its long-term complication. Firstly, we identified two robust ecological clusters which defined severe COVID-19 and post-acute COVID-19. Secondly, these clusters defined by altered multi-biome composition and impaired microbiome functionalities were associated with post-acute COVID-19 syndrome. Lastly, host and microbial factors could predict duration of respiratory viral shedding. For example, 6 host factors and 5 microbial candidates provided high accuracy, hinting at a prognostic potential of microbial markers in determining COVID-19 outcome and consequences.

Several studies demonstrated that gut microbiota composition correlated with severity of COVID-19 infection and persisted months after disease resolution⁷. Gut bacteriome has led to many discoveries of microbiota linked to disease progression of COVID-19⁸, yet there is considerable untapped potential of non-bacterial microorganisms. Disease heterogeneity in COVID-19 given the variability in clinical, immunological inflammatory and human fecal microbiome phenotypes. With the aid of data integration with similarity network fusion approach for multi-kingdom microbiome, we were able to identify specific gut microbiome features that were linked to severity, viral shedding and post-acute complication of COVID-19. Our evaluation on model revealed that including clinical information in addition to gut microbiome significantly improves differentiating capacities to AUC of 0.94 for the COVID-19 cohort. Amongst the microbiome and clinical variables, we found eleven of these factors including bacteria, fungi and viruses significantly associated with cluster patterns and severe status. Using random forest modelling, we observed relationships between features of the different multi-kingdom ecological constituents and patients' clinical features of COVID-19. This embedding approach allowed us to connect these integrated multi-kingdom microbiome signatures to specific clinical measurable features of the disease.

Multi-kingdom microbiome provides new and previously unrecognized targets in that could be considered as an alternative to, or used in combination with, established regimens for prognosis of COVID-19. Particularly in the severe cluster, relationship with other kingdoms such as fungi (Candida glabrata, Candida albucans) and virus are novel and previously unrecognized in COVID-19. The uncovered co-exclusion relationship between pathogenic microorganisms and other species is particularly interesting given the association with disease severity and long-term complications. Assessment of key influential taxa of microorganism in different cluster highlight the relevance of integrative microbiome in precision microbiome. More severe cluster was associated with higher levels of Candida albicans, Pseudomonas phages Pf1 whereas the lower abundance of Bifidobacterium adolescentis. The benefits of targeting influential microbes in an interactome, however, remain unknown and unaddressed by this work, and should be the focus of future studies.

Previous studies reported that blood urea levels, an indication of kidney dysfunction, rose throughout the infection²⁰. Similarly, we found a higher level of urea in patients within the severe cluster than non-severe cluster. Moreover, the functional microbiome revealed that the elevated urea might be explained by gut microbiome-mediated urea nitrogen recycling driven by Klebsiella species such as Klebsiella pneumoniae and Klebsiella variicola. Patients with severe COVID-19 exhibit abnormal bursts of the urea cycle from gut microbiome communities. We found the involvement of gut microbes may hasten the accumulation of blood urea in COVID-19 patients. Klebsiella spp. are considered as the urease-producing and urea-splitting bacteria, which means Klebsiella spp. can produce urease, an enzyme that catalyzes the hydrolysis of urea, forming ammonia and carbon dioxide²¹. Meanwhile, the enhancement of nitro-recycling may in turn cause the increase of serum urea, but the presence of impaired kidney function in COVID-19 may need to be considered as well. Establishing symbiosis to treat uremic toxins is a novel concept, but if proven effective may have a significant impact on the management of patients with COVID-19.

### Supplementation of Beneficial Bacterial to Improve Functional Capacity

In conjunction with our taxonomic profiling, functional profiling of these metagenomes suggested cluster-specific signatures (FIG. 2D, Supplementary Table 5). Among predicted microbiota functions, urea cycle, L-isoleucine degradation I and L-arginine degradation II function were enriched in severe cluster (FIG. 2D, q<0.1, fold change>2). Elevated blood urea nitrogen (BUN) level was also reported to be associated with critical illness and mortality in patients with COVID-19 independent of other covariates and had a robust predictive ability for poor clinical outcome^{14,16}. We next measured blood urea levels in COVID-19 patients. Data indicated that blood urea levels were strongly associated with microbiome urea cycle pathway and presented higher concentration in patients with severe disease status (FIG. 2E, FIG. 2F and FIG. 9A). Next, we investigated whether specific microbiome species are associated with elevated urea in severe COVID-19. To this end, comparing the subclasses pathway and microbial contributors (quantify gene presence and abundance in a species-stratified manner), we saw a pronounced increase in K01940 (argininosuccinate synthase, key enzyme in urea cycle pathway, FIG. 9B) in more severe cluster (FIG. 9C), which was particularly driven by Klebsiella species such as Klebsiella_quasipneumonia, Klebsiella_pneumoniae and Klebsiella_variicola (FIG. 9D). The association of severity with urea was usually explained by the fact that high urea is indication of kidney dysfunction. However, in our cohort, there was no significant difference in other clinical markers of liver and kidney function (total protein, ALP, ALT, creatinine, Supplementary Table 2, Supplementary Table 3) except blood urea. Given the signatures that correlated with disease deterioration, gut-derived uremic toxins into systemic circulation might be one of the explanations for marked increase of urea in severe COVID-19 patients. The enriched L-isoleucine degradation I and L-arginine degradation II, and decreased L-isoleucine biosynthesis IV as well as pyruvate fermentation to acetate and lactate II were further verified by metabolomics sequencing and correlation analysis (FIG. 10). Enriched amino acid degradation and decreased amino acid biosynthesis may be a major source of ammonium for urea cycle.

### Study Methods

### Study Participants

Participants were recruited and consented under Research Ethics Committee (REC) No. 2020.076 and all subjects provided informed consent. This is a cross-sectional and prospective cohort study involving 133 patients with a confirmed diagnosis of COVID-19 (defined as positive RT-PCR test for SARS-CoV-2 in nasopharyngeal swab, deep throat saliva, sputum or tracheal aspirate) hospitalised at three regional hospitals (Prince of Wales Hospital, United Christian Hospital and Yan Chai Hospital) in Hong Kong, China, between 13 Mar. 2020 and 27 Jan. 2021, followed-up to six months. Disease severity at admission was defined based on a clinical score of 1 to 5: (1) asymptomatic, individuals who tested positive for SARS-CoV-2 but who had no symptoms consistent with COVID-19. (2) mild, individuals who had any signs of COVID-19 (e.g., fever, cough, sore throat, malaise, headache, muscle pain) but no radiographic evidence of pneumonia; (3) moderate, if pneumonia was present along with fever and respiratory tract symptoms; (4) severe, if respiratory rate ≥30/min, oxygen saturation 593% when breathing ambient air, or PaO2/FiO2≤300 mm Hg (1 mm Hg=0.133 kPa); or (5) critical, if there was respiratory failure requiring mechanical ventilation, shock, or organ failure requiring intensive care.²² We defined post-acute COVID-19 syndrome (PACS) as at least one persistent symptom or long-term complications of SARS-CoV-2 infection beyond 4 weeks from the onset of symptoms which could not be explained by an alternative diagnosis. We assessed the presence of the 30 most commonly reported symptoms post-COVID at 6 months after illness onset (Supplementary Table 9).

Patients who fulfilled the following criteria were eligible for analyses: (i) 18-70 years of age, (ii) no antibiotic therapy before at least 6 months, during and 6 months after acute infection of SARS-CoV-2 (iii) no gastrointestinal symptoms during acute infection. Written informed consent was obtained from all patients. Dietary data were documented for all COVID-19 patients during the time of hospitalisation (whereby standardised meals were provided by the hospital catering service of each hospital) and individuals with special eating habits such as vegetarians were excluded. After discharge, patients with COVID-19 were advised to continue a diverse and standard Chinese diet that was consistent with habitual daily diets consumed by Hong Kong Chinese. Data on medical history including age, gender, smoking status and comorbidities (i.e., hypertension, diabetes mellitus, hyperlipidemia) were recorded. Laboratory results include liver function tests (total bilirubin, creatine kinase, LDH), renal function (urea, creatinine), complete blood count (i.e., haemoglobin, red blood cell, lymphocyte, monocyte, platelet, polynuclear neutrophil) and CRP were collected.

### Stool Samples

Stool samples were collected at admission from 133 patients and at 3 months and 6 months after discharge (average 3 stool samples per subject). Stool samples from in-hospital patients were collected by hospital staff while discharged patients provided stools on the day of follow-up at 3 month and 6 months after discharge or self-sampled at home and had samples couriered to the hospital within 24 hours of collection. Baseline (stools collected at admission) samples were collected before antibiotic treatment. All samples were collected in tubes containing preservative media (cat. 63700, Norgen Biotek Corp, Ontario Canada) and stored immediately at −80° C. until processing. We have previously shown that data of gut microbiota composition generated from stools collected using this preservative medium is comparable to data obtained from samples that are immediately stored at −80° C.²³.

### Respiratory and Stool SAR-CoV-2 Viral Load

Upper respiratory tract samples (pooled nasopharyngeal and throat swabs), lower respiratory tract samples (sputum and tracheal aspirate), and stool samples from 94 participants were collected at admission. We determined SARS-CoV-2 viral loads in these samples, using real-time reverse-transcriptase-polymerasechain-reaction (RT-PCR) assay with primers and probe targeting the N gene of SARS-CoV-2 designed by US Centers for Disease Control and Prevention²⁴.

### Plasma Cytokine Measurements

Whole blood samples collected in anticoagulant-treated tubes were centrifuged at 2000×g for 10 min and the supernatant was collected. Concentrations of cytokines and chemokines were measured using the MILLIPLEX MAP Human Cytokine/Chemokine Magnetic Bead Panel-Immunology Multiplex Assay (Merck Millipore, Massachusetts, USA) on a Bio-Plex 200 System (Bio-Rad Laboratories, California, USA). The concentration of N-terminal-pro-brain natriuretic peptide (NT-proBNP) was measured using Human NT-proBNP ELISA kits (Abcam, Cambridge, UK).

### Quantification of Fecal Metabolites

The quantification of fecal metabolites was performed by Metware Biotechnology Co., Ltd. (Wuhan, China). Acetic was detected by GC-MS/MS analysis. Agilent 7890B gas chromatography coupled to a 7000D mass spectrometer with a DB-5MS column (30 m length×0.25 mm i.d.×0.25 μm film thickness, J&W Scientific, USA) was used. Helium was used as a carrier gas, at a flow rate of 1.2 mL/min. Injections were made in the splitless mode and the injection volume was 2 μL. The oven temperature was held at 90° C. for 1 min, raised to 100° C. at a rate of 25° C./min, raised to 150° C. at a rate of 20° C./min and held at 150° C. 0.6 min, further raised to 200° C. at a rate of 25° C./min, held at 200° C. 0.5 min. After running for 3 minutes, all samples were analyzed in multiple reaction monitoring modes. The temperature of the injector inlet and transfer line were held at 200° C. and 230° C., respectively. L-isoleucine and L-arginine were detected by LC-MS analysis. LC-ESI-MS/MS system (UPLC, ExionLC AD, website: sciex.com.cn/; MS, QTRAP® 6500+System, website: sciex.com/) was used for analysis. The analytical conditions were as follows, HPLC: column, Waters ACQUITY UPLC HSS T3 C18 (100 mm×2.1 mm i.d., 1.8 μm); solvent system, water with 0.05% formic acid (A), acetonitrile with 0.05% formic acid (B). The gradient was started at 5% B (0-10 min), increased to 95% B (10-11 min), and ramped back to 5% B (11-14 min); flow rate, 0.35 mL/min; temperature, 40° C.; injection volume: 2 μL. The ESI source operation parameters were as follows: an ion source, turbo spray; source temperature 550° C.; ion spray voltage (IS) 5500 V (Positive), −4500 V (Negative); DP and CE for individual MRM transitions were done with further DP and CE optimization.

### Stool DNA Extraction and Sequencing

Detailed methods for extracting bacterial and fungal DNA are described in Zuo et al⁸. The fecal pellet was added to 1 mL of CTAB buffer and vortexed for 30 seconds, then the sample was heated at 95° C. for 5 minutes. After that, the samples were vortexed thoroughly with beads at maximum speed for 15 minutes. Then, 40 μL of proteinase K and 20 μL of RNase A was added to the sample and the mixture was incubated at 70° C. for 10 minutes. The supernatant was then obtained by centrifuging at 13,000 g for 5 minutes and was added to the Maxwell RSC machine for DNA extraction. The total viral DNA was extracted from a fecal sample, using TaKaRa MiniBEST Viral RNA/DNA Extraction Kit (Takara, Japan) following the manufacturer's instructions. Extracted total viral DNA was then purified by the DNA Clean & Concentrator Kits (Zymo Research, CA, USA) to obtain viral DNA, respectively. After the quality control procedures by Qubit 2.0, agarose gel electrophoresis, and Agilent 2100, extracted DNA was subject to DNA libraries construction, completed through the processes of end repairing, adding A to tails, purification and PCR amplification, using Nextera DNA Flex Library Preparation kit (Illumina, San Diego, CA). Libraries were subsequently sequenced on our in-house sequencer Illumina NextSeq 550 (150 base pairs paired-end) at the Center for Microbiota Research, The Chinese University of Hong Kong. Raw sequence data generated for this study are available in the Sequence Read Archive under BioProject accession: PRJNA714459.

### Bioinformatics

Raw sequence data were quality filtered using Trimmomatic V.39 to remove the adaptor, low-quality sequences (quality score <20), reads shorter than 50 base pairs. Contaminating human reads were filtering using Kneaddata (V.0.7.2, website: bitbucket.org/biobakery/kneaddata/wiki/Home, Reference database: GRCh38 p12) with default parameters. Following this, microbiota composition profiles (bacteria and fungi) were inferred from quality-filtered forward reads using MetaPhlAn3 version 3.0.5 and MiCoP. GNU parallel²⁵ was used for parallel analysis jobs to accelerate data processing.

### Bioinformatic Viral Processing

Raw sequence quality was assessed using FASTQC and filtered utilizing Trimmomatic using the following parameters; SLIDINGWINDOW: 4:20, MINLEN: 60 HEADCROP 15; CROP 225. Contaminating human reads were filtering using Kneaddata (Reference database: GRCh38 p12) with default parameters. Megahit²⁶ with default parameters, was chosen to assemble the reads into contigs per sample. Assemblies were subsequently pooled and retained if longer than 1 kb. Bacterial contamination was removed by using an extensive set of inclusion criteria to select viral sequences only. Briefly, contigs were required to fulfill one of the following criteria; 1) Categories 1-6 from VirSorter when run with default parameters and Refseqdb (-db 1)²⁷ positive, 2) circular, 3) greater than 3 kb with no BLASTn alignments to the NT database (January '19) (e-value threshold: 1e-10), 4) a minimum of 2 pVogs with at least 3 per 1 kb²⁸, 5) BLASTn alignments to viral RefSeq database (v.89) (e-value threshold: 1e-10), and 6) less than 3 ribosomal proteins as predicted using the COG database²¹. HMMscan was used to search the pVOGs hmm profile database using predicted protein sequences on VLS with and e-value filter of 1e-5, retaining the top hit in each case. Afterwards, a fasta file combining viral contigs was compiled. This viral database includes the viral contigs recovered by the screening criteria from the bulk metagenomic assemblies. Then the paired reads were mapped to the viral contig database with BWA, using default parameters. The viral operational taxonomic unit (OTU) table of viral abundance was pulled from BWA sam output files by script, and normalized by the number of metagenomic reads. The contigs that were analyzed according to their open reading frames (ORFs). The ORFs on the contigs were predicted using MetaProdigal (Hyatt et al., 2012) (v2.6.3) with the metagenomics procedure (-p meta). To annotate the predicted ORFs, the amino acid sequences of the ORFs were queried by Diamond³⁰ against the viral RefSeq protein (v84) with an E value <10-5 and a bitscore >50. The viral Refseq proteins with the top closest homologies (E value <10-5 and bitscore >50) were considered for each ORF, analogous to a previously reported method³¹.

### Integration and Clustering Analysis of Multi-Biome Data

For each biome dataset, microbes prevalent in at least 5% of patients (that is, n≥7) with an average abundance of 1% were kept for analysis. Integration of bacterial, fungal and viral community data was performed by weighted SNF (WSNF) using an online tool (https://integrative-microbiomics.ntu.edu.sg). Briefly, the respective weights of each biome are assigned based on the richness of the data, as demonstrated by the number of species present in each biome. Using the merged dataset (bacteria, fungi and viruses), the tool generates a corresponding patient similarity network using a spectral clustering algorithm with the default settings (Bray-Curtis), outputting the cluster assignments for each patient. The optimal number of clusters (n=2) was determined using the eigengap method and the value of K nearest neighbours was set based on the optimal silhouette width.

### Random Forest Stratification

R package randomForest v4.6-14 was used to develop a stratification model of patients in different clusters. Four datasets from 133 patients including demographic, blood test, cytokines and multibiome were used separately or in combination (ensemble) to train the model. Machine learning models were first trained on the training set (70%, n=93), and then were applied to the validation set (30%, n=40) to infer the ability of the model to classify new, unseen data. This process was repeated 10 times to obtain a distribution of random forests prediction evaluations on the validation set. For the construction of optimal prediction model in the ensembled data set, the importance value of each feature to the stratification model was evaluated by recursive feature elimination first, and then the selected features are added to the model one by one according to the descending importance value if its Person correlation value with any previous features was less than 0.7. Each time a new feature was added to the model, the performance of the model was re-evaluated using the above training and validation set. The final model was chosen when the best accuracy was achieved.

### Random Forest Regression Analysis for Positive Time Prediction

The random forest regression model was used to regress features from ensembled data set (demographic, blood test, cytokines and multibiome) in the time-series profiling of COVID-19 patients against their SARS-CoV-2019 positive time (Upper respiratory tract) using default parameters of R package randomForest v4.6-14. The RF algorithm, due to its non-parametric assumptions, was applied and used to detect both linear and nonlinear relationships between multiple types of features and positive time, thereby identifying features that discriminate different viral persistent times in COVID-19 patients. Ranked lists of important features in order of reported feature importance were determined over five times 10-fold of the algorithm on the training set (70%, n=93). To estimate the minimal number of top-ranked positive time-discriminatory features required for prediction, the rfcv function implemented in the randomForest package (v4.6-14) was applied over five times 10-fold. A sparse model consisting of the top 10 features was then validated on the validation set (30%, n=40). The predicted positive time was paired with the real positive time for accuracy evaluation, and the accuracy was calculated at different error levels from ±0 to ±5 days.

Co-Occurrence Analysis of Microbial Interaction within COVID-19 Patient Clusters

A weighted ensemble-based co-occurrence analysis along with Reboot was implemented to identify the microbial association networks. Co-occurrence analysis was implemented with statistical significance testing using Reboot as described in Faust et al¹⁹, following the modifications raised by AogAin et al³². The visualization of the interaction network is completed by Cytoscape (3.9.1).

### Statistical Analysis and Inferring Gut Microbiota Composition

Continuous variables were expressed in median (interquartile range) whereas categorical variables were presented as numbers (percentage). Qualitative and quantitative differences between subgroups were analysed using chi-squared or Fisher's exact tests for categorical parameters and Mann-Whitney test for continuous parameters, as appropriate. Odds ratio and adjusted odds ratio (aOR) with 95% confidence interval (CI) were estimated using logistic regression to examine clinical parameters associated with the development of PACS. The site by species counts and relative abundance tables were input into R V.3.5.1 for statistical analysis. Principal Coordinates Analysis (PCoA) was used to visualise the clustering of samples based on their species-level compositional profiles. Associations between gut community composition and patients' parameters were assessed using permutational multivariate analysis of variance (PERMANOVA). Associations of specific microbial species with patient parameters were identified using the linear discriminant analysis effect size (LEfSe) and the multivariate analysis by linear models (MaAsLin2) statistical frameworks implemented in the Huttenhower Lab Galaxy instance (website: huttenhower.sph.harvard.edu/galaxy/). PCoA, PERMANOVA and Procrustes analysis are implemented in the vegan R package V.2.5-7.

### Data Availability Statement

Data are available in a public, open access repository**. Raw sequence data are available in the Sequence Read Archive (SRA) under BioProject accession***.

All patents, patent applications, and other publications, including GenBank Accession Numbers, cited in this application are incorporated by reference in the entirety for all purposes.

### REFERENCES

1. Vabret N, Britton G J, Gruber C, et al. Immunology of COVID-19: current state of the science. Immunity 2020; 52:910-941.
2. Hoffmann M, Kleine-Weber H, Schroeder S, et al. SARS-CoV-2 cell entry depends on ACE2 and TMPRSS2 and is blocked by a clinically proven protease inhibitor. cell 2020; 181:271-280. e8.
3. Lamers M M, Beumer J, Van Der Vaart J, et al. SARS-CoV-2 productively infects human gut enterocytes. Science 2020; 369:50-54.
4. Wang W, Xu Y, Gao R, et al. Detection of SARS-CoV-2 in different types of clinical specimens. Jama 2020; 323:1843-1844.
5. Wölfel R, Corman V M, Guggemos W, et al. Virological assessment of hospitalized patients with COVID-2019. Nature 2020; 581:465-469.
6. Neurath M F, Überla K, Ng S C. Gut as viral reservoir: lessons from gut viromes, HIV and COVID-19. Gut 2021; 70:1605-1608.
7. Yeoh Y K, Zuo T, Lui G C-Y, et al. Gut microbiota composition reflects disease severity and dysfunctional immune responses in patients with COVID-19. Gut 2021; 70:698-706.
8. Zuo T, Zhang F, Lui G C, et al. Alterations in gut microbiota of patients with COVID-19 during time of hospitalization. Gastroenterology 2020; 159:944-955. e8.
9. Ng S C, Tilg H. COVID-19 and the gastrointestinal tract: more than meets the eye. Gut 2020; 69:973-974.
10. Liu Q, Mak J W Y, Su Q, et al. Gut microbiota dynamics in a prospective cohort of patients with post-acute COVID-19 syndrome. Gut 2022.
11. Tay M Z, Poh C M, Rénia L, et al. The trinity of COVID-19: immunity, inflammation and intervention. Nature Reviews Immunology 2020; 20:363-374.
12. Zhou F, Yu T, Du R, et al. Clinical course and risk factors for mortality of adult inpatients with COVID-19 in Wuhan, China: a retrospective cohort study. The lancet 2020; 395:1054-1062.
13. Zhang F, Wan Y, Zuo T, et al. Prolonged impairment of short-chain fatty acid and L-isoleucine biosynthesis in gut microbiome in patients with COVID-19. Gastroenterology 2022; 162:548-561. e4.
14. Cheng A, Hu L, Wang Y, et al. Diagnostic performance of initial blood urea nitrogen combined with D-dimer levels for predicting in-hospital mortality in COVID-19 patients. International journal of antimicrobial agents 2020; 56:106110.
15. Coperchini F, Chiovato L, Rotondi M. Interleukin-6, CXCL10 and infiltrating macrophages in COVID-19-related cytokine storm: Not one for all but all for one! Frontiers in immunology 2021; 12.
16. Huang D, Yang H, Yu H, et al. Blood urea nitrogen to serum albumin ratio (BAR) predicts critical illness in patients with Coronavirus disease 2019 (COVID-19). International journal of general medicine 2021; 14:4711.
17. Huang C, Huang L, Wang Y, et al. 6-month consequences of COVID-19 in patients discharged from hospital: a cohort study. The Lancet 2021.
18. Nalbandian A, Sehgal K, Gupta A, et al. Post-acute COVID-19 syndrome. Nature medicine 2021; 27:601-615.
19. van Kampen J J A, van de Vijver D, Fraaij P L A, et al. Duration and key determinants of infectious virus shedding in hospitalized patients with coronavirus disease-2019 (COVID-19). Nat Commun 2021; 12:267.
20. Abedian S, Wong S H, Van Sommeren S, et al. Explained variance and predictability of inflammatory bowel diseases by genetic risk score in five asian populations (results from the international IBD genetics consortium). Gut 2019; 68:A110-A110.
21. Faust K, Sathirapongsasuti J F, Izard J, et al. Microbial co-occurrence relationships in the human microbiome. PLoS computational biology 2012; 8:e1002606.
22. Wu J, Liu J, Zhao X, et al. Clinical characteristics of imported cases of coronavirus disease 2019 (COVID-19) in Jiangsu Province: a multicenter descriptive study. Clinical Infectious Diseases 2020; 71:706-712.
23. Tange O. Gnu Parallel. DOI: https://doi.org/10.5281/zenodo 2018; 1146014.
24. Li D, Luo R, Liu C-M, et al. MEGAHIT v1. 0: A fast and scalable metagenome assembler driven by advanced methodologies and community practices. Methods 2016; 102:3-11.
25. Roux S, Hallam S J, Woyke T, et al. Viral dark matter and virus-host interactions resolved from publicly available microbial genomes. elife 2015; 4:e08490.
26. Grazziotin A L, Koonin E V, Kristensen D M. Prokaryotic virus orthologous groups (pVOGs): a resource for comparative genomics and protein family annotation. Nucleic acids research 2016:gkw975.
27. Tatusov R L, Galperin M Y, Natale D A, et al. The COG database: a tool for genome-scale analysis of protein functions and evolution. Nucleic acids research 2000; 28:33-36.
28. Buchfink B, Xie C, Huson D H. Fast and sensitive protein alignment using DIAMOND. Nature methods 2015; 12:59-60.
29. Kang D-W, Adams J B, Gregory A C, et al. Microbiota Transfer Therapy alters gut ecosystem and improves gastrointestinal and autism symptoms: an open-label study. Microbiome 2017; 5:10.

SUPPLEMENTARY TABLE 1
Associations between clusters and multi-microbiome

| | coef | stderr | N | N.not.0 | pval | qval |
|---|---|---|---|---|---|---|
| Bacteriome | | | | | | |
| Bfidobacterium_adolescentis | 0.465775 | 0.09973 | 133 | 95 | 7.44E−06 | 0.004555 |
| Eubacterium_hallii | 0.367198 | 0.091892 | 133 | 98 | 0.000108 | 0.022616 |
| Blautia_wexlerae | 0.468885 | 0.121399 | 133 | 115 | 0.000177 | 0.027059 |
| Dorea_longicatena | 0.349049 | 0.0929 | 133 | 94 | 0.000259 | 0.031714 |
| Fusicatenibacter_saccharivorans | 0.453919 | 0.129037 | 133 | 87 | 0.000602 | 0.052597 |
| Ruminococcus gnavus | −0.39554 | 0.119108 | 133 | 96 | 0.001167 | 0.065919 |
| Anaerostipes_hadrus | 0.320559 | 0.095408 | 133 | 103 | 0.001026 | 0.065919 |
| Klebsiella_quasipneumoniae | −0.25553 | 0.078421 | 133 | 28 | 0.001432 | 0.068156 |
| Coprococcus_comes | 0.346629 | 0.10724 | 133 | 70 | 0.001559 | 0.068156 |
| Streptococcus_salivarius | 0.371906 | 0.114278 | 133 | 104 | 0.001451 | 0.068156 |
| Collinsella_stercoris | 0.309937 | 0.098833 | 133 | 88 | 0.002122 | 0.076389 |
| Agathobaculum_butyriciproducens | 0.35295 | 0.112406 | 133 | 100 | 0.002095 | 0.076389 |
| Faecalibacterium_prausnitzii | 0.25575 | 0.085801 | 133 | 94 | 0.003438 | 0.092657 |
| Mycobiome | | | | | | |
| Wickerhamomyces.ciferrii | 0.626072 | 0.098757 | 133 | 61 | 3.55E−09 | 9.60E−08 |
| Aspergillus.flavus | −0.57728 | 0.120471 | 133 | 61 | 4.47E−06 | 6.04E−05 |
| Candida.albicans | −0.41309 | 0.09838 | 133 | 56 | 4.96E−05 | 0.000447 |
| Candida.glabrata | −0.41552 | 0.112496 | 133 | 72 | 0.000325 | 0.002196 |
| Saccharomyces.cerevisiae | −0.21071 | 0.068948 | 133 | 20 | 0.002727 | 0.01227 |
| Aspergillus.niger | −0.37404 | 0.150076 | 133 | 48 | 0.013959 | 0.037689 |
| Virome | | | | | | |
| Nodularia_phage_vB_NspS.kac65v151 | −0.45233 | 0.082476 | 133 | 97 | 2.11E−07 | 0.018123 |
| Clostera_anachoreta_granulovirus | −0.30385 | 0.056166 | 133 | 111 | 2.96E−07 | 0.018123 |
| Mycobacterium_phage_MyraDee | −0.48359 | 0.093138 | 133 | 84 | 7.87E−07 | 0.024118 |
| Pseudomonas_virus_Pf1 | −0.48245 | 0.09646 | 133 | 110 | 1.82E−06 | 0.043436 |
| Beihai_Nido.like_virus_2 | −0.41975 | 0.084533 | 133 | 101 | 2.13E−06 | 0.043436 |
| Streptococcus_phage_SW2 | −0.39297 | 0.082504 | 133 | 102 | 5.05E−06 | 0.061848 |
| Rhodobacter_phage_RcTitan | −0.36232 | 0.076022 | 133 | 94 | 4.99E−06 | 0.061848 |
| Lactococcus_phage_936_group_phage_Phi4 | −0.32132 | 0.067169 | 133 | 111 | 4.63E−06 | 0.061848 |
| Salmonella_phage_FSL_SP.076 | −0.27721 | 0.057489 | 133 | 107 | 3.94E−06 | 0.061848 |
| Gordonia_phage_Yvonnetastic | −0.45063 | 0.095167 | 133 | 111 | 5.68E−06 | 0.063226 |
| Mycobacterium_phage_GenevaB15 | −0.41117 | 0.089594 | 133 | 110 | 1.04E−05 | 0.098125 |
| Acinetobacter_phage_SH.Ab_15497 | −0.36513 | 0.079286 | 133 | 110 | 9.75E−06 | 0.098125 |

SUPPLEMENTARY TABLE 2
Microbiome function pathway profile in two clusters.

| pathway | coef | stderr | N | N.not.0 | pval | qval | log2fold | log10q |
|---|---|---|---|---|---|---|---|---|
| PWY_4984 | −0.47693 | 0.075614 | 120 | 49 | 5.34E−09 | 1.05E−03 | −2.12306 | −2.97855 |
| AST_PWY | −0.39231 | 0.07119 | 120 | 57 | 2.19E−07 | 7.24E−03 | −1.06064 | −2.14045 |
| ILEUDEG-PWY | −0.42507 | 0.067958 | 120 | 56 | 6.87E−09 | 1.05E−03 | −1.04653 | −2.97855 |
| PWY_6969 | −0.15957 | 0.046711 | 120 | 109 | 0.000877 | 0.007866 | −0.99788 | −2.10423 |
| GLYCOLYSIS_TCA_GLYOX_BYPASS | −0.38472 | 0.063628 | 120 | 79 | 1.85E−08 | 1.37E−03 | −0.98794 | −2.86426 |
| PWY_5971 | −0.15625 | 0.045385 | 120 | 98 | 0.000802 | 0.007435 | −0.9816 | −2.12872 |
| PWY_7312 | −0.13665 | 0.091113 | 120 | 57 | 0.136395 | 0.345486 | −0.98025 | −0.46157 |
| PWY_5525 | −0.46381 | 0.071732 | 120 | 63 | 2.48E−09 | 1.05E−03 | −0.97927 | −2.97855 |
| PWY_7211 | −0.20123 | 0.065076 | 120 | 95 | 0.002488 | 0.018662 | −0.97639 | −1.72905 |
| P161_PWY | −0.32632 | 0.052598 | 120 | 114 | 8.76E−09 | 1.05E−03 | −0.95557 | −2.97855 |
| PWY_6519 | −0.16325 | 0.03302 | 120 | 118 | 2.61E−06 | 5.32E−05 | −0.94917 | −4.27399 |
| PWY_5104 | −0.18298 | 0.051504 | 120 | 115 | 0.000552 | 0.005409 | −0.93781 | −2.26686 |
| PYRIDOXSYN_PWY | −0.21874 | 0.048512 | 120 | 117 | 1.57E−05 | 0.000247 | −0.93743 | −3.60807 |
| PWY_1269 | −0.20298 | 0.041869 | 120 | 118 | 3.91E−06 | 7.65E−05 | −0.93338 | −4.11615 |
| NAD_BIOSYNTHESIS_II | −0.14331 | 0.060022 | 120 | 108 | 0.018577 | 0.095132 | −0.93281 | −1.02167 |
| PWY_7315 | −0.19629 | 0.062977 | 120 | 104 | 0.002304 | 0.017555 | −0.93242 | −1.7556 |
| PWY_6630 | −0.21808 | 0.045273 | 120 | 42 | 4.45E−06 | 8.21E−02 | −0.9185 | −1.08576 |
| ARG.POLYAMINE_SYN | −0.19584 | 0.035144 | 120 | 115 | 1.66E−07 | 5.68E−06 | −0.91836 | −5.24598 |
| PWY_6285 | −0.3014 | 0.058535 | 120 | 73 | 1.08E−06 | 2.60E−02 | −0.91592 | −1.58562 |
| PWY0_862 | −0.13156 | 0.034028 | 120 | 119 | 0.000183 | 0.002089 | −0.88709 | −2.68006 |
| PWY_6282 | −0.12785 | 0.033457 | 120 | 119 | 0.000215 | 0.002427 | −0.88546 | −2.61493 |
| PWY_6284 | −0.15898 | 0.061861 | 120 | 98 | 0.011436 | 0.069045 | −0.87617 | −1.16087 |
| DENOVOPURINE2_PWY | −0.174 | 0.051865 | 120 | 45 | 0.001073 | 0.009038 | −0.87178 | −2.04392 |
| PWY_7664 | −0.12873 | 0.033041 | 120 | 119 | 0.000164 | 0.001903 | −0.86843 | −2.72066 |
| PRPP_PWY | −0.1745 | 0.050924 | 120 | 45 | 0.000846 | 0.007734 | −0.86296 | −2.11158 |
| FASYN_ELONG_PWY | −0.12632 | 0.032212 | 120 | 119 | 0.000149 | 0.001802 | −0.85235 | −2.74429 |
| BIOTIN_BIOSYNTHESIS_PWY | −0.17052 | 0.036058 | 120 | 118 | 6.40E−06 | 0.000112 | −0.85062 | −3.95186 |
| TCA | −0.12246 | 0.031439 | 120 | 116 | 0.000164 | 0.001903 | −0.8479 | −2.72066 |
| PWY_5863 | −0.3178 | 0.07598 | 120 | 65 | 5.62E−05 | 0.00074 | −0.84374 | −3.13096 |
| PWY_5918 | −0.32037 | 0.071317 | 120 | 95 | 1.68E−05 | 0.00026 | −0.83854 | −3.58572 |
| PWY0_881 | −0.16927 | 0.074758 | 120 | 53 | 0.025417 | 0.119025 | −0.83673 | −0.92436 |
| GLYCOLYSIS_E_D | −0.16744 | 0.036147 | 120 | 118 | 9.53E−06 | 0.000163 | −0.83244 | −3.78687 |
| PWY0_166 | −0.35396 | 0.079176 | 120 | 75 | 1.83E−05 | 0.00027 | −0.82884 | −3.56868 |
| PWY_7094 | −0.33335 | 0.078174 | 120 | 50 | 4.11E−05 | 0.563789 | −0.82334 | −0.24888 |
| PWY_5747 | −0.4521 | 0.083077 | 120 | 69 | 2.98E−07 | 9.53E−03 | −0.80649 | −2.02085 |
| PWY0_845 | −0.19133 | 0.04322 | 120 | 117 | 2.17E−05 | 0.000316 | −0.79695 | −3.49996 |
| HCAMHPDEG_PWY | −0.41821 | 0.072846 | 120 | 64 | 7.67E−08 | 3.88E−03 | −0.79614 | −2.41166 |
| PWY_6690 | −0.41821 | 0.072846 | 120 | 64 | 7.67E−08 | 3.88E−03 | −0.79614 | −2.41166 |
| PWY_5189 | −0.30552 | 0.06673 | 120 | 98 | 1.18E−05 | 0.000193 | −0.79055 | −3.71341 |
| REDCITCYC | −0.21041 | 0.063104 | 120 | 84 | 0.001148 | 0.009586 | −0.78505 | −2.01835 |
| PWY_5862 | −0.25508 | 0.056893 | 120 | 85 | 1.74E−05 | 0.000265 | −0.78119 | −3.57756 |
| PWY_7388 | −0.12309 | 0.032948 | 120 | 119 | 0.000292 | 0.00308 | −0.7769 | −2.51144 |
| PWY0_1261 | −0.16657 | 0.036251 | 120 | 118 | 1.11E−05 | 0.000187 | −0.76405 | −3.72884 |
| PWY_7204 | −0.39113 | 0.072228 | 120 | 63 | 3.36E−07 | 1.02E−02 | −0.76223 | −1.99011 |
| FAO_PWY | −0.4385 | 0.069176 | 120 | 67 | 4.59E−09 | 1.05E−03 | −0.75501 | −2.97855 |
| P122_PWY | −0.19939 | 0.059334 | 120 | 54 | 0.001054 | 0.008955 | −0.74424 | −2.04794 |
| P441_PWY | −0.11632 | 0.029672 | 120 | 118 | 0.00015 | 0.001802 | −0.70596 | −2.74429 |
| PWY_5837 | −0.23864 | 0.063196 | 120 | 97 | 0.000253 | 0.002763 | −0.70262 | −2.55867 |
| PWY_5855 | −0.37881 | 0.07607 | 120 | 58 | 2.24E−06 | 4.67E−02 | −0.70078 | −1.3305 |
| PWY_5856 | −0.37881 | 0.07607 | 120 | 58 | 2.24E−06 | 4.67E−02 | −0.70078 | −1.3305 |
| PWY_5857 | −0.37881 | 0.07607 | 120 | 58 | 2.24E−06 | 4.67E−02 | −0.70078 | −1.3305 |
| PWY_5989 | −0.10583 | 0.031363 | 120 | 119 | 0.001007 | 0.008633 | −0.70033 | −2.06385 |
| PWY_6708 | −0.36707 | 0.073515 | 120 | 58 | 2.11E−06 | 4.67E−02 | −0.69514 | −1.3305 |
| POLYISOPRENSYN_PWY | −0.13193 | 0.059128 | 120 | 115 | 0.027581 | 0.12669 | −0.68503 | −0.89726 |
| P461_PWY | −0.12693 | 0.040875 | 120 | 116 | 0.002389 | 0.018061 | −0.67673 | −1.74326 |
| PWY_5845 | −0.24475 | 0.05472 | 120 | 85 | 1.81E−05 | 0.00027 | −0.67429 | −3.56868 |
| P108_PWY | −0.13253 | 0.053795 | 120 | 111 | 0.015223 | 0.08351 | −0.67149 | −1.07826 |
| FUC_RHAMCAT_PWY | −0.1137 | 0.044568 | 120 | 113 | 0.012039 | 0.071746 | −0.6652 | −1.1442 |
| PWYO_1277 | −0.40265 | 0.071021 | 120 | 62 | 1.06E−07 | 4.26E−03 | −0.64158 | −2.37077 |
| PWY_5690 | −0.08506 | 0.030226 | 120 | 117 | 0.005749 | 0.038594 | −0.63843 | −1.41348 |
| GALACT_GLUCUROCAT_PWY | −0.09997 | 0.038647 | 120 | 117 | 0.010922 | 0.066362 | −0.62702 | −1.17808 |
| PWY_6859 | −0.0993 | 0.062254 | 120 | 115 | 0.113434 | 0.311133 | −0.61578 | −0.50705 |
| FUCCAT_PWY | −0.10061 | 0.04332 | 120 | 116 | 0.021955 | 0.107534 | −0.61092 | −0.96845 |
| PWY_7269 | −0.39851 | 0.100508 | 120 | 52 | 0.000127 | 0.001568 | −0.6101 | −2.80472 |
| PWY0_1479 | −0.1036 | 0.049176 | 120 | 116 | 0.037303 | 0.153647 | −0.59591 | −0.81348 |
| FERMENTATION_PWY | −0.09729 | 0.046506 | 120 | 114 | 0.038628 | 0.157131 | −0.5894 | −0.80374 |
| SULFATE_CYS_PWY | −0.35821 | 0.06643 | 120 | 105 | 3.72E−07 | 1.08E−05 | −0.58856 | −4.96628 |
| PWY0_1061 | −0.36069 | 0.070042 | 120 | 54 | 1.08E−06 | 2.60E−05 | −0.58389 | −4.58562 |
| PWY_6892 | −0.13266 | 0.060588 | 120 | 109 | 0.030562 | 0.135183 | −0.57303 | −0.86908 |
| GALACTUROCAT_PWY | −0.08831 | 0.041801 | 120 | 117 | 0.036784 | 0.15282 | −0.5726 | −0.81582 |
| PWY_5920 | −0.37813 | 0.072015 | 120 | 68 | 6.95E−07 | 1.80E−02 | −0.56748 | −1.74425 |
| HEME_BIOSYNTHESIS_II | −0.1876 | 0.05653 | 120 | 106 | 0.001209 | 0.009839 | −0.55905 | −2.00703 |
| PWY_5464 | −0.05868 | 0.049628 | 120 | 98 | 0.239468 | 0.476948 | −0.55742 | −0.32153 |
| PWY_5861 | −0.24852 | 0.059777 | 120 | 94 | 6.19E−05 | 0.000803 | −0.5524 | −3.09523 |
| PWY_7013 | −0.0083 | 0.055099 | 120 | 112 | 0.880453 | 0.943343 | −0.54143 | −0.02533 |
| PWY_5840 | −0.21565 | 0.057727 | 120 | 92 | 0.000292 | 0.00308 | −0.53886 | −2.51144 |
| PWY_7187 | −0.26239 | 0.069495 | 120 | 44 | 0.000253 | 0.002763 | −0.5363 | −2.55867 |
| HISDEG_PWY | −0.08464 | 0.03548 | 120 | 119 | 0.018677 | 0.095132 | −0.51541 | −1.02167 |
| PWY4LZ_257 | −0.28981 | 0.06124 | 120 | 113 | 6.32E−06 | 0.000112 | −0.51138 | −3.95186 |
| ARGDEG_PWY | −0.33342 | 0.062752 | 120 | 56 | 5.27E−07 | 1.45E−02 | −0.49907 | −1.83961 |
| ORNARGDEG_PWY | −0.33342 | 0.062752 | 120 | 56 | 5.27E−07 | 1.45E−02 | −0.49907 | −1.83961 |
| PWY_5656 | −0.45086 | 0.080286 | 120 | 68 | 1.36E−07 | 5.02E−03 | −0.49514 | −2.29892 |
| PWY_4702 | −0.41063 | 0.075881 | 120 | 89 | 3.41E−07 | 1.02E−02 | −0.49346 | −1.99011 |
| PWY0_1415 | −0.34108 | 0.064713 | 120 | 82 | 6.36E−07 | 1.70E−02 | −0.48595 | −1.77036 |
| PWY_7663 | −0.07211 | 0.023814 | 120 | 120 | 0.003032 | 0.022054 | −0.48565 | −1.65652 |
| PWY_5994 | −0.2063 | 0.064193 | 120 | 39 | 0.001696 | 0.013239 | −0.48381 | −1.87813 |
| PWY_6507 | −0.08269 | 0.037798 | 120 | 118 | 0.030698 | 0.135183 | −0.4825 | −0.86908 |
| PWY_7282 | −0.12226 | 0.030717 | 120 | 118 | 0.00012 | 0.001521 | −0.47806 | −2.81777 |
| ECASYN_PWY | −0.35023 | 0.067349 | 120 | 58 | 8.66E−07 | 2.19E−02 | −0.47608 | −1.66006 |
| PWY4FS_8 | −0.09903 | 0.026622 | 120 | 120 | 0.000309 | 0.00322 | −0.47558 | −2.4922 |
| PWY4FS_7 | −0.09894 | 0.026625 | 120 | 120 | 0.000313 | 0.003229 | −0.47514 | −2.49089 |
| PWY_5083 | −0.45571 | 0.081679 | 120 | 70 | 1.60E−07 | 5.68E−03 | −0.46755 | −2.24598 |
| PWY_5675 | −0.58657 | 0.095227 | 120 | 77 | 1.08E−08 | 1.15E−03 | −0.46683 | −2.93784 |
| PWY_5791 | −0.28745 | 0.05957 | 120 | 65 | 4.29E−06 | 8.08E−02 | −0.46572 | −1.09259 |
| SALVADEHYPOX_PWY | −0.05002 | 0.041094 | 120 | 116 | 0.226031 | 0.466644 | −0.46303 | −0.33101 |
| PWY_5838 | −0.23514 | 0.057042 | 120 | 94 | 7.08E−05 | 0.000906 | −0.46001 | −3.04295 |
| PWY_7385 | −0.33424 | 0.079149 | 120 | 66 | 4.82E−05 | 0.000652 | −0.45757 | −3.18603 |
| HEXITOLDEGSUPER_PWY | −0.0878 | 0.029964 | 120 | 117 | 0.004082 | 0.027991 | −0.4572 | −1.55298 |
| THISYN_PWY | −0.11854 | 0.045428 | 120 | 109 | 0.010267 | 0.062778 | −0.45058 | −1.20219 |
| PWY0_1298 | −0.22087 | 0.045008 | 120 | 116 | 3.04E−06 | 6.09E−05 | −0.44723 | −4.21542 |
| PWY_5173 | −0.17957 | 0.065795 | 120 | 109 | 0.007337 | 0.047591 | −0.4442 | −1.32247 |
| PHOSLIPSYN_PWY | −0.09763 | 0.024554 | 120 | 119 | 0.000122 | 0.001523 | −0.44224 | −2.81743 |
| PWY0_1297 | −0.09231 | 0.036063 | 120 | 120 | 0.011759 | 0.070554 | −0.43883 | −1.15148 |
| PWY_7242 | −0.06239 | 0.039237 | 120 | 118 | 0.114518 | 0.311436 | −0.4247 | −0.50663 |
| PWY_7539 | −0.08853 | 0.028863 | 120 | 119 | 0.002688 | 0.019853 | −0.42417 | −1.70218 |
| HOMOSER_METSYN_PWY | −0.07452 | 0.033329 | 120 | 120 | 0.027279 | 0.126509 | −0.41482 | −0.89788 |
| PWY_6147 | −0.08737 | 0.02909 | 120 | 119 | 0.003269 | 0.023421 | −0.41253 | −1.6304 |
| PWY_5897 | −0.20995 | 0.056853 | 120 | 97 | 0.00034 | 0.003395 | −0.41061 | −2.46914 |
| PWY_5898 | −0.20995 | 0.056853 | 120 | 97 | 0.00034 | 0.003395 | −0.41061 | −2.46914 |
| PWY_5899 | −0.20995 | 0.056853 | 120 | 97 | 0.00034 | 0.003395 | −0.41061 | −2.46914 |
| ARGININE_SYN4_PWY | −0.09114 | 0.051908 | 120 | 116 | 0.08177 | 0.259989 | −0.40598 | −0.58504 |
| POLYAMINSYN3_PWY | −0.0458 | 0.061933 | 120 | 78 | 0.461045 | 0.6747 | −0.40489 | −0.17089 |
| PWY_7392 | −0.06653 | 0.059953 | 120 | 115 | 0.269403 | 0.509107 | −0.39173 | −0.29319 |
| PWY_6588 | 0.023222 | 0.074157 | 120 | 106 | 0.754735 | 0.877174 | −0.39169 | −0.05691 |
| HEMESYN2_PWY | −0.13257 | 0.041218 | 120 | 112 | 0.001683 | 0.013239 | −0.37941 | −1.87813 |
| PWY_5723 | −0.43 | 0.068856 | 120 | 89 | 7.20E−09 | 1.05E−03 | −0.3787 | −2.97855 |
| GLUDEG_I_PWY | −0.32312 | 0.0679 | 120 | 100 | 5.66E−06 | 0.000103 | −0.3635 | −3.98904 |
| PWY66_398 | −0.18722 | 0.056549 | 120 | 78 | 0.00124 | 0.010007 | −0.35726 | −1.99972 |
| PWY_7115 | −0.08423 | 0.053501 | 120 | 110 | 0.11811 | 0.316719 | −0.34976 | −0.49933 |
| RUMP_PWY | −0.1004 | 0.083895 | 120 | 76 | 0.233833 | 0.471572 | −0.34822 | −0.32645 |
| RHAMCAT_PWY | −0.0651 | 0.021802 | 120 | 120 | 0.003451 | 0.024357 | −0.34781 | −1.61338 |
| GLUCOSE1PMETAB_PWY | −0.07855 | 0.062336 | 120 | 115 | 0.210183 | 0.447396 | −0.34754 | −0.34931 |
| COLANSYN_PWY | −0.08878 | 0.028314 | 120 | 119 | 0.002173 | 0.016691 | −0.34484 | −1.77752 |
| GLUCUROCAT_PWY | −0.06171 | 0.038784 | 120 | 117 | 0.114287 | 0.311436 | −0.3431 | −0.50663 |
| PWY_5659 | −0.0573 | 0.017935 | 120 | 120 | 0.001801 | 0.013941 | −0.33338 | −1.85572 |
| KETOGLUCONMET_PWY | −0.55998 | 0.092079 | 120 | 74 | 1.57E−08 | 1.37E−03 | −0.33012 | −2.86426 |
| PWY_5345 | −0.32548 | 0.065 | 120 | 105 | 1.99E−06 | 4.55E−05 | −0.32911 | −4.34211 |
| PWY_5973 | −0.04716 | 0.021404 | 120 | 120 | 0.029557 | 0.132592 | −0.32018 | −0.87748 |
| PWY0_1241 | −0.22236 | 0.051306 | 120 | 115 | 3.13E−05 | 0.000443 | −0.31634 | −3.35315 |
| PWY_5030 | −0.03615 | 0.048533 | 120 | 118 | 0.457903 | 0.672152 | −0.3103 | −0.17253 |
| PWY_7323 | −0.08996 | 0.029931 | 120 | 118 | 0.003252 | 0.023421 | −0.30808 | −1.6304 |
| PWY_7220 | −0.03571 | 0.021314 | 120 | 120 | 0.096544 | 0.282259 | −0.30461 | −0.54935 |
| PWY_7222 | −0.03571 | 0.021314 | 120 | 120 | 0.096544 | 0.282259 | −0.30461 | −0.54935 |
| PWY_7184 | −0.25974 | 0.075175 | 120 | 98 | 0.000769 | 0.00724 | −0.29578 | −2.14028 |
| PWY_5154 | −0.0577 | 0.019992 | 120 | 118 | 0.004654 | 0.03169 | −0.29238 | −1.49908 |
| PWY_5022 | −0.25387 | 0.059155 | 120 | 108 | 3.70E−05 | 0.000514 | −0.28846 | −3.28877 |
| PENTOSE_P_PWY | −0.04887 | 0.019991 | 120 | 120 | 0.016005 | 0.086321 | −0.28037 | −1.06388 |
| PWY_6731 | −0.0699 | 0.060786 | 120 | 112 | 0.252538 | 0.488751 | −0.28036 | −0.31091 |
| PWY_6803 | −0.49082 | 0.080359 | 120 | 87 | 1.38E−08 | 1.33E−03 | −0.2791 | −2.87648 |
| PWY0_1338 | −0.50792 | 0.081045 | 120 | 64 | 6.48E−09 | 1.05E−03 | −0.27383 | −2.97855 |
| METSYN_PWY | −0.06116 | 0.029815 | 120 | 120 | 0.042485 | 0.166991 | −0.26016 | −0.77731 |
| ORNDEG_PWY | −0.40292 | 0.066539 | 120 | 63 | 1.77E−08 | 1.37E−03 | −0.24968 | −2.86426 |
| P42_PWY | −0.09904 | 0.073586 | 120 | 109 | 0.180977 | 0.407836 | −0.22942 | −0.38951 |
| PWY_5347 | −0.05767 | 0.028361 | 120 | 120 | 0.044274 | 0.171522 | −0.22932 | −0.76568 |
| CITRULBIO_PWY | −0.02682 | 0.058367 | 120 | 114 | 0.646731 | 0.825614 | −0.22798 | −0.08322 |
| PWY_5121 | −0.02841 | 0.04569 | 120 | 118 | 0.535235 | 0.735627 | −0.21889 | −0.13334 |
| P105_PWY | −0.38651 | 0.062261 | 120 | 68 | 8.60E−09 | 1.05E−03 | −0.21789 | −2.97855 |
| NAGLIPASYN_PWY | −0.25994 | 0.05063 | 120 | 116 | 1.15E−06 | 2.70E−05 | −0.21786 | −4.5683 |
| PWY_6606 | −0.05768 | 0.031236 | 120 | 117 | 0.067368 | 0.230976 | −0.21578 | −0.63643 |
| PWY0_162 | −0.02822 | 0.028852 | 120 | 119 | 0.329987 | 0.567913 | −0.21051 | −0.24572 |
| PWY_6595 | −0.07226 | 0.060575 | 120 | 104 | 0.23534 | 0.471572 | −0.20487 | −0.32645 |
| PWY_4988 | −0.02901 | 0.058945 | 120 | 114 | 0.623588 | 0.808255 | −0.20305 | −0.09245 |
| MET_SAM_PWY | −0.05689 | 0.029395 | 120 | 120 | 0.055368 | 0.200579 | −0.20251 | −0.69771 |
| PWY_6901 | −0.05984 | 0.024349 | 120 | 119 | 0.015473 | 0.084399 | −0.19646 | −1.07366 |
| PWY_5860 | −0.28715 | 0.062729 | 120 | 67 | 1.19E−05 | 0.193458 | −0.19507 | −0.71341 |
| PWY_6125 | −0.02267 | 0.017636 | 120 | 120 | 0.201206 | 0.436022 | −0.19287 | −0.36049 |
| PWY_6353 | −0.02906 | 0.034363 | 120 | 116 | 0.399482 | 0.632843 | −0.19262 | −0.1987 |
| PWY30_355 | 0.021338 | 0.066817 | 120 | 109 | 0.750033 | 0.875158 | −0.19048 | −0.05791 |
| GLYOXYLATE_BYPASS | −0.39932 | 0.06989 | 120 | 89 | 8.70E−08 | 4.18E−03 | −0.18707 | −2.37928 |
| GALACTARDEG_PWY | −0.37788 | 0.064123 | 120 | 102 | 3.80E−08 | 2.28E−03 | −0.18616 | −2.64258 |
| GLUCARGALACTSUPER_PWY | −0.37788 | 0.064123 | 120 | 102 | 3.80E−08 | 2.28E−03 | −0.18616 | −2.64258 |
| PWY_2723 | −0.0597 | 0.064777 | 120 | 114 | 0.358613 | 0.601097 | −0.17737 | −0.22106 |
| PWY_7228 | −0.02021 | 0.016091 | 120 | 120 | 0.211654 | 0.448538 | −0.16975 | −0.3482 |
| TCA_GLYOX_BYPASS | −0.4143 | 0.068719 | 120 | 79 | 2.01E−08 | 1.38E−03 | −0.16821 | −2.86071 |
| METHGLYUT_PWY | −0.21136 | 0.054103 | 120 | 106 | 0.000158 | 0.00187 | −0.16734 | −2.72823 |
| PWY66_389 | −0.33897 | 0.10327 | 120 | 85 | 0.001361 | 0.010795 | −0.16708 | −1.96677 |
| PWY_6612 | −0.0683 | 0.076567 | 120 | 105 | 0.374226 | 0.611488 | −0.167 | −0.21361 |
| PWY_5913 | −0.0364 | 0.053322 | 120 | 116 | 0.496222 | 0.708888 | −0.16607 | −0.14942 |
| PWY_5367 | −0.12163 | 0.07953 | 120 | 98 | 0.128906 | 0.334507 | −0.15871 | −0.47559 |
| PWY0_781 | −0.05625 | 0.0454 | 120 | 114 | 0.217824 | 0.457573 | −0.15415 | −0.33954 |
| PWY_6305 | −0.07836 | 0.030867 | 120 | 118 | 0.01245 | 0.073325 | −0.15052 | −1.13475 |
| PWY_7254 | −0.36127 | 0.063586 | 120 | 84 | 1.01E−07 | 4.26E−03 | −0.14514 | −2.37077 |
| PWY_6126 | −0.01644 | 0.015007 | 120 | 120 | 0.275534 | 0.514615 | −0.143 | −0.28852 |
| GLYCOCAT_PWY | −0.05389 | 0.061728 | 120 | 114 | 0.384451 | 0.623435 | −0.13844 | −0.20521 |
| FOLSYN_PWY | −0.06571 | 0.075402 | 120 | 105 | 0.385288 | 0.623738 | −0.13599 | −0.205 |
| GLUCONEO_PWY | −0.0468 | 0.020436 | 120 | 120 | 0.023819 | 0.112092 | −0.13453 | −0.95043 |
| GLUCARDEG_PWY | −0.37611 | 0.065068 | 120 | 98 | 6.40E−08 | 3.62E−03 | −0.13233 | −2.44184 |
| PWY_6545 | −0.25484 | 0.07301 | 120 | 97 | 0.000683 | 0.006487 | −0.1305 | −2.18794 |
| PWY_6703 | −0.03389 | 0.024997 | 120 | 120 | 0.177759 | 0.403777 | −0.12718 | −0.39386 |
| PWY66_409 | −0.04445 | 0.033916 | 120 | 117 | 0.192532 | 0.422953 | −0.10859 | −0.37371 |
| PWY_6531 | −0.23192 | 0.06163 | 120 | 105 | 0.000265 | 0.002856 | −0.10837 | −2.54423 |
| PWY_7117 | −0.03008 | 0.048814 | 120 | 116 | 0.538995 | 0.737087 | −0.10699 | −0.13248 |
| PWY_5896 | −0.25844 | 0.053405 | 120 | 65 | 4.05E−06 | 7.77E−02 | −0.1038 | −1.10931 |
| PWY0_1586 | −0.01705 | 0.015705 | 120 | 120 | 0.279944 | 0.518245 | −0.10344 | −0.28547 |
| GLCMANNANAUT_PWY | −0.02564 | 0.021274 | 120 | 120 | 0.230638 | 0.471572 | −0.10217 | −0.32645 |
| PWY_7328 | −0.03989 | 0.058305 | 120 | 116 | 0.495262 | 0.708572 | −0.09994 | −0.14962 |
| ASPASN_PWY | −0.02249 | 0.014172 | 120 | 120 | 0.115301 | 0.3118 | −0.09941 | −0.50612 |
| PWY_7229 | −0.01045 | 0.011817 | 120 | 120 | 0.378454 | 0.616836 | −0.09079 | −0.20983 |
| THREOCAT_PWY | −0.30792 | 0.071058 | 120 | 49 | 3.14E−05 | 0.44346 | −0.08809 | −0.35315 |
| PWY_7197 | −0.01288 | 0.020771 | 120 | 120 | 0.536395 | 0.735627 | −0.08576 | −0.13334 |
| PWY_5850 | −0.27631 | 0.060514 | 120 | 67 | 1.25E−05 | 0.199259 | −0.08427 | −0.70058 |
| PWY_7208 | −0.01398 | 0.0175 | 120 | 120 | 0.425863 | 0.655173 | −0.0805 | −0.18364 |
| PWY_7383 | 0.045556 | 0.056973 | 120 | 116 | 0.425575 | 0.655173 | −0.07566 | −0.18364 |
| PWY_4041 | −0.00724 | 0.026909 | 120 | 120 | 0.788376 | 0.892501 | −0.06711 | −0.04939 |
| PWY_841 | −0.01055 | 0.012329 | 120 | 120 | 0.394041 | 0.630466 | −0.06083 | −0.20034 |
| UNINTEGRATED | −0.01235 | 0.003658 | 120 | 120 | 0.000999 | 0.008633 | −0.0603 | −2.06385 |
| PWY_5484 | −0.03191 | 0.023315 | 120 | 120 | 0.173714 | 0.40226 | −0.0523 | −0.39549 |
| SO4ASSIM_PWY | −0.41013 | 0.072103 | 120 | 105 | 9.78E−08 | 4.26E−03 | −0.05173 | −2.37077 |
| PWY_821 | −0.32458 | 0.057451 | 120 | 69 | 1.17E−07 | 4.47E−03 | −0.04999 | −2.34928 |
| ARGORNPROST_PWY | −0.11264 | 0.053558 | 120 | 83 | 0.037611 | 0.153647 | −0.04908 | −0.81348 |
| POLYAMSYN_PWY | −0.22097 | 0.038947 | 120 | 115 | 1.04E−07 | 4.26E−06 | −0.04827 | −5.37077 |
| PWY_7199 | −0.01056 | 0.011757 | 120 | 120 | 0.371143 | 0.610621 | −0.04584 | −0.21423 |
| P185_PWY | 0.075031 | 0.046822 | 120 | 118 | 0.111768 | 0.308326 | −0.03224 | −0.51099 |
| PWY_7332 | −0.04503 | 0.08813 | 120 | 74 | 0.610387 | 0.799416 | −0.02028 | −0.09723 |
| GLYCOLYSIS | −0.02649 | 0.02255 | 120 | 120 | 0.242574 | 0.480285 | −0.0171 | −0.3185 |
| PWY_6897 | −0.01889 | 0.016288 | 120 | 120 | 0.248513 | 0.488664 | −0.01603 | −0.31099 |
| PWY66_399 | 0.083465 | 0.056809 | 120 | 115 | 0.144478 | 0.356553 | −0.01339 | −0.44788 |
| PWY_6608 | −0.01246 | 0.028638 | 120 | 119 | 0.664284 | 0.834786 | −0.01314 | −0.07842 |
| PWY_5497 | −0.20928 | 0.061358 | 120 | 99 | 0.000892 | 0.00793 | −0.00846 | −2.10073 |
| PWY_5695 | −0.00335 | 0.009441 | 120 | 120 | 0.72304 | 0.861664 | −0.00617 | −0.06466 |
| P4_PWY | −0.04595 | 0.04519 | 120 | 116 | 0.311319 | 0.551414 | −0.00378 | −0.25852 |
| PWY_5265 | −0.04163 | 0.079172 | 120 | 59 | 0.600012 | 0.787977 | −0.00112 | −0.10349 |
| PWY_7210 | −0.25126 | 0.07606 | 120 | 74 | 0.001271 | 0.010165 | −0.00069 | −1.9929 |
| PANTO_PWY | −0.01326 | 0.016536 | 120 | 120 | 0.424231 | 0.655173 | 0.020602 | −0.18364 |
| PPGPPMET_PWY | −0.01454 | 0.056677 | 120 | 114 | 0.798033 | 0.898941 | 0.044192 | −0.04627 |
| FASYN_INITIAL_PWY | 0.00354 | 0.009892 | 120 | 120 | 0.721114 | 0.861664 | 0.050695 | −0.06466 |
| PWY_5667 | −0.01178 | 0.01778 | 120 | 120 | 0.50894 | 0.719561 | 0.05613 | −0.14293 |
| PWY0_1319 | −0.01143 | 0.017734 | 120 | 120 | 0.520379 | 0.72611 | 0.056739 | −0.139 |
| PWY_5676 | 0.003483 | 0.035716 | 120 | 114 | 0.922477 | 0.969251 | 0.059754 | −0.01356 |
| PWY_7456 | −0.00206 | 0.044697 | 120 | 114 | 0.96325 | 0.986894 | 0.062664 | −0.00573 |
| PWY_5005 | −0.03042 | 0.057555 | 120 | 108 | 0.598086 | 0.787603 | 0.066866 | −0.10369 |
| PWY_5384 | −0.03757 | 0.048219 | 120 | 118 | 0.437416 | 0.657448 | 0.068747 | −0.18214 |
| PANTOSYN_PWY | −0.00586 | 0.015054 | 120 | 120 | 0.697713 | 0.854358 | 0.079457 | −0.06836 |
| PWY_2941 | 0.031807 | 0.033435 | 120 | 120 | 0.343438 | 0.585357 | 0.079906 | −0.23258 |
| NONOXIPENT_PWY | 0.00693 | 0.019493 | 120 | 120 | 0.722853 | 0.861664 | 0.094293 | −0.06466 |
| RIBOSYN2_PWY | −0.00453 | 0.020429 | 120 | 120 | 0.824733 | 0.913199 | 0.097716 | −0.03943 |
| PWY_5188 | 0.012562 | 0.023523 | 120 | 118 | 0.59433 | 0.78481 | 0.101488 | −0.10524 |
| PWY_6124 | 0.018272 | 0.008448 | 120 | 120 | 0.032607 | 0.141642 | 0.113684 | −0.84881 |
| PWY_241 | −0.01352 | 0.052893 | 120 | 114 | 0.798747 | 0.898941 | 0.114716 | −0.04627 |
| PWY_7111 | 0.006955 | 0.009813 | 120 | 120 | 0.479925 | 0.691784 | 0.115659 | −0.16003 |
| PYRIDNUCSYN_PWY | 0.001197 | 0.015722 | 120 | 119 | 0.939458 | 0.97606 | 0.117739 | −0.01052 |
| PWY_6609 | 0.012416 | 0.009384 | 120 | 120 | 0.1884 | 0.420615 | 0.120974 | −0.37612 |
| THRESYN_PWY | 0.009637 | 0.010722 | 120 | 120 | 0.370614 | 0.610621 | 0.122683 | −0.21423 |
| PWY_7198 | 0.037526 | 0.02759 | 120 | 120 | 0.176424 | 0.403777 | 0.124606 | −0.39386 |
| ANAEROFRUCAT_PWY | −0.00904 | 0.021655 | 120 | 120 | 0.67718 | 0.839533 | 0.125887 | −0.07596 |
| PWY_6123 | 0.019827 | 0.00836 | 120 | 120 | 0.019357 | 0.09729 | 0.129327 | −1.01193 |
| PWY_7234 | 0.025514 | 0.038932 | 120 | 118 | 0.513538 | 0.722365 | 0.130207 | −0.14124 |
| TEICHOICACID_PWY | 0.023149 | 0.028119 | 120 | 118 | 0.412054 | 0.64676 | 0.131156 | −0.18926 |
| ILEUSYN_PWY | 0.010841 | 0.009437 | 120 | 120 | 0.253031 | 0.488751 | 0.134345 | −0.31091 |
| VALSYN_PWY | 0.010841 | 0.009437 | 120 | 120 | 0.253031 | 0.488751 | 0.134345 | −0.31091 |
| THISYNARA_PWY | 0.030904 | 0.033121 | 120 | 120 | 0.352739 | 0.595131 | 0.138406 | −0.22539 |
| PWY_3001 | 0.007685 | 0.012864 | 120 | 120 | 0.551418 | 0.746966 | 0.143751 | −0.1267 |
| X1CMET2_PWY | 0.01697 | 0.008065 | 120 | 120 | 0.037511 | 0.153647 | 0.14531 | −0.81348 |
| PWY_7003 | 0.069897 | 0.066278 | 120 | 104 | 0.2938 | 0.535271 | 0.14748 | −0.27143 |
| PWY_6168 | 0.003652 | 0.020133 | 120 | 120 | 0.85637 | 0.926635 | 0.15176 | −0.03309 |
| PWY_6700 | 0.015649 | 0.010867 | 120 | 120 | 0.15255 | 0.370754 | 0.152645 | −0.43091 |
| GOLPDLCAT_PWY | −0.00221 | 0.053599 | 120 | 116 | 0.96716 | 0.989844 | 0.154226 | −0.00443 |
| DTDPRHAMSYN_PWY | 0.015675 | 0.01229 | 120 | 120 | 0.20469 | 0.439602 | 0.156806 | −0.35694 |
| CALVIN_PWY | 0.011142 | 0.013738 | 120 | 120 | 0.419022 | 0.650908 | 0.157032 | −0.18648 |
| PWY_6385 | 0.015029 | 0.00752 | 120 | 120 | 0.047985 | 0.182801 | 0.15772 | −0.73802 |
| COA_PWY | 0.015159 | 0.008375 | 120 | 120 | 0.072885 | 0.24211 | 0.160565 | −0.61599 |
| ANAGLYCOLYSIS_PWY | 0.019767 | 0.008359 | 120 | 120 | 0.019711 | 0.098556 | 0.160747 | −1.00632 |
| PWY_6549 | 0.026193 | 0.040318 | 120 | 115 | 0.517194 | 0.724064 | 0.163415 | −0.14022 |
| PWY_2942 | 0.01696 | 0.009354 | 120 | 120 | 0.072413 | 0.24211 | 0.172512 | −0.61599 |
| TRNA_CHARGING_PWY | 0.019634 | 0.007041 | 120 | 120 | 0.006189 | 0.04126 | 0.173882 | −1.38447 |
| PWY_6122 | 0.019743 | 0.00784 | 120 | 120 | 0.013159 | 0.075169 | 0.174875 | −1.12396 |
| PWY_6277 | 0.019743 | 0.00784 | 120 | 120 | 0.013159 | 0.075169 | 0.174875 | −1.12396 |
| PWY_3841 | 0.024006 | 0.006727 | 120 | 120 | 0.000523 | 0.005177 | 0.175622 | −2.2859 |
| PWY_4242 | 0.012699 | 0.010325 | 120 | 120 | 0.221207 | 0.461249 | 0.176911 | −0.33606 |
| P164_PWY | 0.042056 | 0.046057 | 120 | 115 | 0.363076 | 0.603032 | 0.179509 | −0.21966 |
| PWY_6121 | 0.021714 | 0.00762 | 120 | 120 | 0.005182 | 0.035034 | 0.180654 | −1.45552 |
| PWY_6936 | 0.002471 | 0.026556 | 120 | 120 | 0.926026 | 0.969449 | 0.182502 | −0.01347 |
| DAPLYSINESYN_PWY | −0.01849 | 0.040642 | 120 | 120 | 0.649989 | 0.828672 | 0.184765 | −0.08162 |
| COA_PWY_1 | 0.020531 | 0.008417 | 120 | 120 | 0.016239 | 0.086825 | 0.188248 | −1.06136 |
| UNMAPPED | 0.036573 | 0.010752 | 120 | 120 | 0.00092 | 0.008101 | 0.189825 | −2.09145 |
| PWY_1042 | 0.016144 | 0.01084 | 120 | 120 | 0.139131 | 0.349648 | 0.191742 | −0.45637 |
| PWY0_1296 | 0.011846 | 0.021278 | 120 | 120 | 0.578792 | 0.770652 | 0.193208 | −0.11314 |
| PEPTIDOGLYCANSYN_PWY | 0.02139 | 0.007901 | 120 | 120 | 0.007809 | 0.050312 | 0.19367 | −1.29833 |
| BRANCHED_CHAIN_AA_SYN_PWY | 0.013683 | 0.01431 | 120 | 120 | 0.340944 | 0.582396 | 0.197726 | −0.23478 |
| PWY_6387 | 0.021879 | 0.007924 | 120 | 120 | 0.006698 | 0.044347 | 0.19982 | −1.35313 |
| PWY_724 | 0.010083 | 0.014305 | 120 | 120 | 0.482313 | 0.694184 | 0.200001 | −0.15853 |
| PWY_5686 | 0.023082 | 0.008576 | 120 | 120 | 0.008162 | 0.051943 | 0.201424 | −1.28447 |
| HISTSYN_PWY | 0.020636 | 0.012154 | 120 | 120 | 0.092208 | 0.274054 | 0.20321 | −0.56216 |
| PWY_5097 | 0.021608 | 0.009793 | 120 | 120 | 0.029313 | 0.132208 | 0.214769 | −0.87874 |
| NONMEVIPP_PWY | 0.014587 | 0.013741 | 120 | 120 | 0.290639 | 0.533142 | 0.214772 | −0.27316 |
| PWY_5107 | 0.015997 | 0.015622 | 120 | 120 | 0.307966 | 0.546483 | 0.219925 | −0.26242 |
| PWY_6386 | 0.02501 | 0.008239 | 120 | 120 | 0.002965 | 0.021727 | 0.223377 | −1.66301 |
| PWY_7221 | 0.029154 | 0.007715 | 120 | 120 | 0.00025 | 0.002763 | 0.236626 | −2.55867 |
| COMPLETE_ARO_PWY | 0.02508 | 0.011823 | 120 | 120 | 0.036026 | 0.151028 | 0.24328 | −0.82094 |
| CENTFERM_PWY | 0.009478 | 0.051516 | 120 | 107 | 0.854356 | 0.925713 | 0.249374 | −0.03352 |
| PWY_6163 | 0.031166 | 0.008908 | 120 | 120 | 0.000664 | 0.006379 | 0.250281 | −2.19525 |
| PWY_6590 | 0.009668 | 0.050909 | 120 | 107 | 0.849708 | 0.924852 | 0.251436 | −0.03393 |
| ARO_PWY | 0.025501 | 0.012484 | 120 | 120 | 0.043347 | 0.169157 | 0.252275 | −0.77171 |
| UDPNAGSYN_PWY | 0.016497 | 0.024215 | 120 | 120 | 0.497052 | 0.709019 | 0.254092 | −0.14934 |
| PWY_7560 | 0.016239 | 0.016589 | 120 | 120 | 0.329668 | 0.567913 | 0.259328 | −0.24572 |
| PWY_6270 | 0.017266 | 0.016278 | 120 | 120 | 0.291007 | 0.533142 | 0.259776 | −0.27316 |
| PWY_7219 | 0.03529 | 0.008386 | 120 | 120 | 5.09E−05 | 0.000679 | 0.263711 | −3.16794 |
| SER_GLYSYN_PWY | 0.02426 | 0.013601 | 120 | 120 | 0.077094 | 0.250883 | 0.264719 | −0.60053 |
| PWY_6737 | 0.022683 | 0.019743 | 120 | 120 | 0.25295 | 0.488751 | 0.292743 | −0.31091 |
| COBALSYN_PWY | 0.019562 | 0.027601 | 120 | 117 | 0.479912 | 0.691784 | 0.302167 | −0.16003 |
| OANTIGEN_PWY | 0.033395 | 0.022532 | 120 | 120 | 0.141034 | 0.352586 | 0.302758 | −0.45274 |
| PYRIDNUCSAL_PWY | 0.0135 | 0.055353 | 120 | 114 | 0.80775 | 0.900627 | 0.313491 | −0.04546 |
| TRPSYN_PWY | 0.039922 | 0.015288 | 120 | 120 | 0.01021 | 0.062778 | 0.313673 | −1.20219 |
| PWY_6317 | 0.029998 | 0.025149 | 120 | 120 | 0.235366 | 0.471572 | 0.32966 | −0.32645 |
| PWY66_422 | 0.041789 | 0.017526 | 120 | 119 | 0.018729 | 0.095132 | 0.33423 | −1.02167 |
| PWY_6151 | 0.042914 | 0.012207 | 120 | 120 | 0.000627 | 0.006083 | 0.341925 | −2.2159 |
| PWY_6527 | 0.033397 | 0.027112 | 120 | 120 | 0.220509 | 0.461196 | 0.356719 | −0.33611 |
| PWY_5505 | 0.053327 | 0.0492 | 120 | 114 | 0.28067 | 0.518245 | 0.357881 | −0.28547 |
| GLUTORN_PWY | 0.034449 | 0.018142 | 120 | 119 | 0.060065 | 0.213566 | 0.361889 | −0.67047 |
| PWY_7400 | 0.041007 | 0.016404 | 120 | 119 | 0.013823 | 0.076704 | 0.365368 | −1.11518 |
| ARGSYN_PWY | 0.041274 | 0.016428 | 120 | 119 | 0.013365 | 0.075235 | 0.365742 | −1.12358 |
| PWY_7237 | 0.051909 | 0.040111 | 120 | 106 | 0.198192 | 0.430462 | 0.367654 | −0.36607 |
| ARGSYNBSUB_PWY | 0.032102 | 0.026352 | 120 | 120 | 0.225616 | 0.466644 | 0.375734 | −0.33101 |
| HSERMETANA_PWY | 0.057642 | 0.026832 | 120 | 120 | 0.033773 | 0.144742 | 0.382295 | −0.83941 |
| PWY_6470 | 0.078548 | 0.052677 | 120 | 112 | 0.138645 | 0.349648 | 0.383055 | −0.45637 |
| PWY_7357 | 0.032774 | 0.021294 | 120 | 120 | 0.126495 | 0.330886 | 0.391777 | −0.48032 |
| PWY_7209 | 0.044123 | 0.072407 | 120 | 93 | 0.543473 | 0.740432 | 0.392064 | −0.13051 |
| P125_PWY | 0.097725 | 0.082479 | 120 | 87 | 0.2385 | 0.476007 | 0.473698 | −0.32239 |
| PWY_4981 | 0.100479 | 0.047333 | 120 | 119 | 0.035895 | 0.151028 | 0.483663 | −0.82094 |
| GLYCOGENSYNTH_PWY | 0.051376 | 0.021799 | 120 | 120 | 0.020108 | 0.099971 | 0.497473 | −1.00013 |
| PWY_6471 | 0.129229 | 0.049309 | 120 | 117 | 0.009948 | 0.062001 | 0.503269 | −1.2076 |
| PWY_1861 | 0.070379 | 0.055124 | 120 | 116 | 0.204242 | 0.439602 | 0.520943 | −0.35694 |
| PWY_6863 | 0.100541 | 0.055529 | 120 | 108 | 0.072794 | 0.24211 | 0.526171 | −0.61599 |
| PWY_622 | 0.096197 | 0.070924 | 120 | 94 | 0.177623 | 0.403777 | 0.647855 | −0.39386 |
| P124_PWY | 0.150762 | 0.079565 | 120 | 106 | 0.060604 | 0.213897 | 0.799075 | −0.66979 |
| METH_ACETATE_PWY | 0.242942 | 0.072922 | 120 | 106 | 0.001159 | 0.009592 | 0.856327 | −2.0181 |
| LACTOSECAT_PWY | 0.138663 | 0.058454 | 120 | 118 | 0.01933 | 0.09729 | 0.883531 | −1.01193 |
| PWY_7196 | 0.164804 | 0.08458 | 120 | 57 | 0.053771 | 0.196275 | 1.100805 | −0.70713 |
| PWY_5103 | 0.2302 | 0.066892 | 120 | 102 | 0.000805 | 0.007435 | 1.28092 | −2.12872 |
| PWY_5100 | 0.061207 | 0.03149 | 120 | 120 | 0.005435 | 0.019764 | 2.063156 | −1.70412 |

SUPPLEMENTARY TABLE 3
Comparison of clinical parameters in two clusters.

| Plasma | Cluster 1 | | Cluster 2 | | p_valuie | Reference | Units | Marker |
|---|---|---|---|---|---|---|---|---|
| RBC | 4.6 | (4.3-4.9) | 4.8 | (4.4-5.1) | 0.23512698 | 3.70-4.95 | *10\hat{ }12/ | |
| | | | | | | | L | |
| Haemoglobin | 13.6 | (13.0-14.0) | 14.0 | (13.0-15.2) | 0.19891173 | 11.9-15.1 | g/dL | |
| HCT | 0.40 | (0.38-0.43) | 0.4 | (0.39-0.45) | 0.3524869 | 0.35-0.44 | L/L | |
| MCV | 86.8 | (85.0-90.3) | 86.4 | (84.0-59.7) | 0.77752136 | 83.0-98.0 | fL | |
| MCH | 29.6 | (29.0-31.2) | 29.4 | (28.7-30.9) | 0.79780979 | 28.0-34.0 | pg | |
| MCHC | 34.0 | (33.5-34.6) | 34.0 | (33.7-34.5) | 0.98684172 | 32.9-35.3 | g/dL | |
| RDW | 13.2 | (12.8-13.6) | 13.0 | (12.4-13.5) | 0.24127502 | 12.0-14.7 | % | |
| Platelet | 245.2 | (172.0-285.0) | 217.7 | (163.5-265.5) | 0.16709056 | 150-384 | *10\hat{ }9/L | |
| MPV | 8.41 | (7.8-8.9) | 8.8 | (8.1-9.3) | 0.08483178 | 7.3-10.5 | fL | |
| WBC | 6.3 | (4.0-6.9) | 6.0 | (4.5-6.7) | 0.71597757 | 4.0-9.7 | *10\hat{ }9/L | |
| NEU | 66.0 | (56.0-76.9) | 64.9 | (55.5-75.0) | 0.67797767 | 41-73 | % | |
| LYM | 23.2 | (13.0-30.9) | 23.9 | (15.5-31.0) | 0.70193388 | 15-44 | % | |
| MON | 10.0 | (7.0-13.8) | 9.5 | (7.5-11.0) | 0.47280419 | 4-11 | % | |
| EOS | 1.8 | (0.0-2.8) | 1.35 | (0-2.0) | 0.51887727 | 1-9 | % | |
| BAS | 0.3 | (0.0-1.7) | 0.4 | (0-1.0) | 0.63571512 | 0-1 | % | |
| Sodium | 137.3 | (134.75-140) | 138.6 | (136.5-141.0) | 0.07430289 | 136-145 | mmol/L | |
| Potassium | 3.8 | (3.6-4.1) | 3.85 | (3.6-4.1) | 0.77518249 | 3.5-5.1 | mmol/L | |
| Urea | 5.1 | (3.9-5.5) | 4.0 | (3.2-4.8) | 0.00673344 | 2.8-8.1 | mmol/L | ** |
| Creatinine | 73.3 | (60.0-88.6) | 80.2 | (68.0-86.0) | 0.25162591 | 44-80 | umol/L | |
| Total_protein | 72.5 | (67.0-78.0) | 71.3 | (67.0-76.5) | 0.43102168 | 66-87 | g/L | |
| Albumin | 33.7 | (28.0-39.3) | 35.3 | (31.5-39) | 0.2843774 | 35-52 | g/L | |
| Total_Bilirubin | 13.2 | (7.0-17.4) | 12.8 | (6.5-14.5) | 0.82045237 | <22 | umol/L | |
| Total_ALP | 60.6 | (49.0-70.0) | 65.9 | (50.5-73.0) | 0.21008231 | 35-104 | IU/L | |
| ALT | 34.8 | (17.0-47.0) | 29.7 | (15.3-37.8) | 0.28203606 | <33 | IU/L | |
| Calcium | 2.2 | (2.155-2.335) | 2.2 | (2.1-2.3) | 0.62026646 | 2.15-2.55 | mmol/L | |
| Adj. calcium | 2.3 | (2.255-2.36) | 2.3 | (2.2-2.3) | 0.40285155 | 2.15-2.55 | mmol/L | |
| Phosphate | 0.9 | (0.84-1.085) | 0.9 | (0.8-1.0) | 0.62812375 | 0.81-1.45 | mmol/L | |
| CPK | 105.6 | (49.75-118.75) | 129.8 | (54.8-101.3) | 0.51860276 | 26-192 | U/L | |
| LDH | 249.2 | (185.25-291.75) | 210.6 | (160.0-228.5) | 0.01128617 | 103-199 | U/L | * |
| AST/GDT | 34.2 | (25.5-39.5) | 26.8 | (20.5-33.0) | 0.03113769 | <35 | U/L | * |
| CRP | 19.9 | (3.9-34.2) | 11.1 | (0.85-17.6) | 0.02729318 | <9.9 | mg/L | * |

Supplementary Table 4 Comparison of cytokines in two clusters.

| Cytokines | Cluster 1 | | Cluster 2 | | p | Marker |
|---|---|---|---|---|---|---|
| CXCL10 | 2583.2 | (686.1-2868.0) | 1326.3 | (327.5-2068.7) | 0.03 | * |
| ACE_2 | 16.7 | (2.0-3.5) | 15.3 | (2.0-18.6) | 0.14 | |
| IL_1b | 2.1 | (0.8-1.76) | 3.5 | (0.8-4.0) | 0.15 | |
| IL_6 | 15.0 | (0.9-13.5) | 26.6 | (2.5-16.18) | 0.30 | |
| TNF_a | 42.5 | (6.88-11.32) | 27.5 | (5.0-11.5) | 0.44 | |
| CCL2 | 409.9 | (213.2-386.0) | 321.9 | (177.7-351.7) | 0.48 | |
| IL_10 | 9.5 | (1.8-13.8) | 8.3 | (3.8-14.0) | 0.56 | |
| CXCL8 | 54.7 | (4.1-13.0) | 40.2 | (4.2-19.7) | 0.70 | |
| NT_proBNP | 96.8 | (41.9-97.3) | 105.1 | (52.4-130.6) | 0.75 | |
| IL_12p70 | 4.7 | (0.6-4.68) | 4.4 | (0.6-7.0) | 0.87 | |

SUPPLEMENTARY TABLE 5
AUC value of different datasets.

| | AUC (Average | p_value | | | | | |
|---|---|---|---|---|---|---|---|
| | of repeating | | | | | Ensemble_Predic- | Ensemble_Predic- |
| Dataset | 10 times) | Demographics | Blood_test | Cytokines | Multibiome | tion | tion_top11 |
| Demographics | 0.52854 | . . . | 0.167394611 | 0.060796976 | 3.62082E−07 | 1.07357E−09 | 1.9211E−09 |
| Blood_test | 0.59861 | | . . . | 0.853719559 | 1.79939E−05 | 4.41895E−08 | 7.22411E−08 |
| Cytokines | 0.6062 | | | . . . | 2.82267E−07 | 3.33074E−11 | 1.28179E−10 |
| Multibiome | 0.83822 | | | | . . . | 0.000967769 | 0.001792313 |
| Ensemble_Predic- | 0.93607 | | | | | . . . | 0.997633202 |
| tion | | | | | | | |
| Ensemble_Predic- | 0.93601 | | | | | | . . . |
| tion_top11 | | | | | | | |

Supplementary Table 6 Top eleven contributors
to the random forest classification model.

| | | | | 95% Confidence | |
|---|---|---|---|---|---|
| | | | | Interal | |
| | | Std. | | Lower | Upper |
| | AUC | Error | p | Bound | Bound |
| Merged | 0.981 | 0.006 | 6.49E−10 | 0.94 | 0.982 |
| Age | 0.57 | 0.05 | 0.162 | 0.473 | 0.668 |
| Viral load | 0.477 | 0.09 | 0.796 | 0.301 | 0.653 |
| LDH | 0.654 | 0.057 | 0.011 | 0.541 | 0.766 |
| CRP | 0.586 | 0.063 | 0.182 | 0.461 | 0.71 |
| CXCL10 | 0.629 | 0.065 | 0.053 | 0.502 | 0.756 |
| Bifidobacterium | 0.588 | 0.049 | 0.08 | 0.491 | 0.685 |
| adolescentis | | | | | |
| Faecalibacterium prausnitzii | 0.648 | 0.048 | 0.003 | 0.553 | 0.743 |
| Blautia wexlerae | 0.657 | 0.047 | 0.002 | 0.565 | 0.75 |
| Candida albicans | 0.672 | 0.046 | 0.001 | 0.581 | 0.763 |
| Aspergillus niger | 0.733 | 0.113 | 0.065 | 0.512 | 0.954 |
| Pseudomonas virus Pf1 | 0.763 | 0.042 | 1.81E−07 | 0.68 | 0.845 |

SUPPLEMENTARY TABLE 7
Characteristics of core network of gut multi-microbiome.

| | | | | Clus- | | | |
|---|---|---|---|---|---|---|---|
| | Aver- | Between- | Close- | tering | | | |
| | ageShort- | nessCen- | nessCen- | Coeffi- | | Eccen- | IsSin- |
| | estPathLength | trality | trality | cient | Degree | tricity | gleNode |
| Cluster 1 | | | | | | | |
| B_Actinomyces_graevenitzii | 2.304094 | 0.001454 | 0.43401 | 0.351373 | 51 | 3 | FALSE |
| B_Actinomyces_odontolyticus | 2.250292 | 7.87E−04 | 0.444387 | 0.366327 | 47 | 3 | FALSE |
| B_Bacteroides_dorei | 2.166082 | 7.82E−04 | 0.461663 | 0.408805 | 54 | 3 | FALSE |
| B_Bifidobacterium_dentium | 2.184795 | 3.44E−04 | 0.457709 | 0.527282 | 64 | 3 | FALSE |
| B_Clostridium_spiroforme | 1.933333 | 0.00174 | 0.517241 | 0.45876 | 106 | 3 | FALSE |
| B_Clostridium_symbiosum | 2.08538 | 0.001415 | 0.479529 | 0.298797 | 47 | 3 | FALSE |
| B_Enterococcus_faecalis | 2.209357 | 9.30E−04 | 0.45262 | 0.304422 | 49 | 3 | FALSE |
| B_Fusobacterium_ulcerans | 1.955556 | 0.002283 | 0.511364 | 0.419956 | 96 | 3 | FALSE |
| B_Klebsiella_pneumoniae | 2.104094 | 8.38E−04 | 0.475264 | 0.419643 | 64 | 3 | FALSE |
| B_Klebsiella_variicola | 2.239766 | 0.001359 | 0.446475 | 0.388539 | 54 | 3 | FALSE |
| F_Candida.albicans | 2.460819 | 1.10E−04 | 0.406369 | 0.45098 | 18 | 4 | FALSE |
| F_Grosmannia.clavigera | 2.425731 | 1.21E−04 | 0.412247 | 0.320261 | 18 | 3 | FALSE |
| F_Moesziomyces.antarcticus | 2.330994 | 4.61E−04 | 0.429002 | 0.322689 | 35 | 4 | FALSE |
| F_Saccharomyces.cerevisiae | 2.487719 | 1.95E−04 | 0.401975 | 0.337662 | 22 | 3 | FALSE |
| F_Sordaria.macrospora | 2.298246 | 3.46E−04 | 0.435115 | 0.249012 | 23 | 3 | FALSE |
| V_Acinetobacter_phage_Ab105.3phi | 1.651462 | 0.010405 | 0.605524 | 0.222045 | 305 | 3 | FALSE |
| V_Aeromonas_phage_Asfd_1 | 1.738012 | 0.005946 | 0.57537 | 0.270257 | 237 | 3 | FALSE |
| V_Bacillus_phage_0305phi8.36 | 1.659649 | 0.011767 | 0.602537 | 0.20583 | 293 | 3 | FALSE |
| V_Bacillus_phage_proCM3 | 1.650292 | 0.011755 | 0.605953 | 0.204186 | 304 | 3 | FALSE |
| V_Clostridium_phage_c.st | 1.725146 | 0.009089 | 0.579661 | 0.221127 | 241 | 3 | FALSE |
| V_Hokovirus_HKV1 | 1.650292 | 0.016618 | 0.605953 | 0.176589 | 300 | 3 | FALSE |
| V_Klebsiella_phage_ST11.OXA245phi3.2 | 1.697076 | 0.00935 | 0.589249 | 0.191304 | 263 | 3 | FALSE |
| V_Klebsiella_phage_ST11.VIM1phi8.2 | 1.748538 | 0.006429 | 0.571906 | 0.249313 | 231 | 3 | FALSE |
| V_Klebsiella_phage_ST13.OXA48phi12.1 | 1.702924 | 0.009156 | 0.587225 | 0.185019 | 262 | 3 | FALSE |
| V_Prokaryotic_dsDNA_virus_sp. | 1.716959 | 0.008883 | 0.582425 | 0.208974 | 252 | 3 | FALSE |
| Cluster 2 | | | | | | | |
| B_Megamonas_hypermegale | 1.73 | 0.003296 | 0.578035 | 0.191898 | 258 | 3 | FALSE |
| B_Megamonas_funiformis | 1.74375 | 0.003219 | 0.573477 | 0.198047 | 240 | 3 | FALSE |
| B_Asaccharobacter_celatus | 1.82 | 0.001648 | 0.549451 | 0.216299 | 176 | 3 | FALSE |
| B_Ruminococcus bromii | 1.9 | 4.42E−04 | 0.526316 | 0.289247 | 128 | 3 | FALSE |
| B_Blautia_wexlerae | 1.90375 | 0.001101 | 0.525279 | 0.327584 | 127 | 3 | FALSE |
| B_Paraprevotella clara | 1.945 | 7.62E−04 | 0.514139 | 0.326061 | 100 | 3 | FALSE |
| B_Bacteroides_cellulosilyticus | 1.97 | 0.001025 | 0.507614 | 0.341392 | 91 | 3 | FALSE |
| B_Coprococcus_catus | 1.95375 | 4.24E−04 | 0.511836 | 0.319459 | 89 | 3 | FALSE |
| B_Clostridium_spiroforme | 2.00875 | 5.99E−04 | 0.497822 | 0.272727 | 77 | 3 | FALSE |
| B_Erysipelatoclostridium_ramosum | 1.985 | 5.45E−04 | 0.503778 | 0.34018 | 75 | 3 | FALSE |
| F_Candida.glabrata | 1.49875 | 0.014044 | 0.667223 | 0.310592 | 420 | 3 | FALSE |
| F_Wickerhamomyces.ciferrii | 1.8875 | 0.001565 | 0.529801 | 0.280969 | 138 | 3 | FALSE |
| F_Saccharomyces.cerevisiae | 2.0275 | 0.003645 | 0.493218 | 0.176623 | 56 | 3 | FALSE |
| F_Candida.dubliniensis | 2.08375 | 2.27E−05 | 0.479904 | 0.428205 | 40 | 4 | FALSE |
| F_Aspergillus.niger | 2.1175 | 3.81E−04 | 0.472255 | 0.322689 | 35 | 4 | FALSE |
| V_Bacillus_phage_Basilisk | 1.41375 | 0.004473 | 0.707339 | 0.389063 | 484 | 3 | FALSE |
| V_Streptococcus_phage_Javan575 | 1.45625 | 0.004337 | 0.686695 | 0.394531 | 452 | 3 | FALSE |
| V_Streptococcus_phage_Javan235 | 1.46625 | 0.004861 | 0.682012 | 0.367292 | 447 | 3 | FALSE |
| V_Escherichia_virus_122 | 1.4825 | 0.004631 | 0.674536 | 0.32186 | 436 | 3 | FALSE |
| V_Gordonia_phage_Stormageddon | 1.5 | 0.004848 | 0.666667 | 0.398042 | 416 | 3 | FALSE |
| V_Xanthomonas_phage_OP2 | 1.51875 | 0.003313 | 0.658436 | 0.307829 | 409 | 3 | FALSE |
| V_Clostridium_phage_phiMMP04 | 1.52375 | 0.003833 | 0.656276 | 0.348042 | 401 | 3 | FALSE |
| V_Paenibacillus_phage_phiERICV | 1.525 | 0.00215 | 0.655738 | 0.392048 | 398 | 3 | FALSE |
| V_Streptococcus_satel- | 1.52875 | 0.004746 | 0.654129 | 0.366543 | 398 | 3 | FALSE |
| lite_phage_Javan593 | | | | | | | |
| V_Clostridium_phage_PhiS63 | 1.52875 | 0.001747 | 0.654129 | 0.433205 | 396 | 3 | FALSE |

| | Neighbor- | NumberOf | NumberOf | PartnerOf | | |
|---|---|---|---|---|---|---|
| | hoodCon- | Direct- | Undirect- | Multi | | select- |
| | nectivity | edEdges | edEdges | EdgedNodePairs | Radiality | ed |
| Cluster 1 | | | | | | |
| B_Actinomyces_graevenitzii | 46.5098 | 0 | 51 | 0 | 0.995724 | FALSE |
| B_Actinomyces_odontolyticus | 59.57447 | 0 | 47 | 0 | 0.995901 | FALSE |
| B_Bacteroides_dorei | 76 | 0 | 54 | 0 | 0.996177 | FALSE |
| B_Bifidobacterium_dentium | 73.6875 | 0 | 64 | 0 | 0.996115 | FALSE |
| B_Clostridium_spiroforme | 162.3113 | 0 | 106 | 0 | 0.99694 | FALSE |
| B_Clostridium_symbiosum | 104.4255 | 0 | 47 | 0 | 0.996441 | FALSE |
| B_Enterococcus_faecalis | 62.06122 | 0 | 49 | 0 | 0.996035 | FALSE |
| B_Fusobacterium_ulcerans | 147.4896 | 0 | 96 | 0 | 0.996867 | FALSE |
| B_Klebsiella_pneumoniae | 79.4375 | 0 | 64 | 0 | 0.99638 | FALSE |
| B_Klebsiella_variicola | 62.5 | 0 | 54 | 0 | 0.995935 | FALSE |
| F_Candida.albicans | 64.05556 | 0 | 18 | 0 | 0.99521 | FALSE |
| F_Grosmannia.clavigera | 68.33333 | 0 | 18 | 0 | 0.995325 | FALSE |
| F_Moesziomyces.antarcticus | 69.8 | 0 | 35 | 0 | 0.995636 | FALSE |
| F_Saccharomyces.cerevisiae | 50.95455 | 0 | 22 | 0 | 0.995122 | FALSE |
| F_Sordaria.macrospora | 70.34783 | 0 | 23 | 0 | 0.995743 | FALSE |
| V_Acinetobacter_phage_Ab105.3phi | 109.7475 | 0 | 305 | 0 | 0.997864 | FALSE |
| V_Aeromonas_phage_Asfd_1 | 118.6203 | 0 | 237 | 0 | 0.99758 | FALSE |
| V_Bacillus_phage_0305phi8.36 | 106.8225 | 0 | 293 | 0 | 0.997837 | FALSE |
| V_Bacillus_phage_proCM3 | 106.5066 | 0 | 304 | 0 | 0.997868 | FALSE |
| V_Clostridium_phage_c.st | 108.8963 | 0 | 241 | 0 | 0.997622 | FALSE |
| V_Hokovirus_HKV1 | 99.24 | 0 | 300 | 0 | 0.997868 | FALSE |
| V_Klebsiella_phage_ST11.OXA245phi3.2 | 102.6882 | 0 | 263 | 0 | 0.997715 | FALSE |
| V_Klebsiella_phage_ST11.VIM1phi8.2 | 114.9524 | 0 | 231 | 0 | 0.997546 | FALSE |
| V_Klebsiella_phage_ST13.OXA48phi12.1 | 102.3282 | 0 | 262 | 0 | 0.997695 | FALSE |
| V_Prokaryotic_dsDNA_virus_sp. | 108.1349 | 0 | 252 | 0 | 0.997649 | FALSE |
| Cluster 2 | | | | | | |
| B_Megamonas_hypermegale | 203.4806 | 0 | 258 | 0 | 0.998492 | FALSE |
| B_Megamonas_funiformis | 202.5375 | 0 | 240 | 0 | 0.998463 | FALSE |
| B_Asaccharobacter_celatus | 201.6875 | 0 | 176 | 0 | 0.998306 | FALSE |
| B_Ruminococcus bromii | 215.8828 | 0 | 128 | 0 | 0.99814 | FALSE |
| B_Blautia_wexlerae | 217.2362 | 0 | 127 | 0 | 0.998133 | FALSE |
| B_Paraprevotella clara | 190.13 | 0 | 100 | 0 | 0.998048 | FALSE |
| B_Bacteroides_cellulosilyticus | 191.7253 | 0 | 91 | 0 | 0.997996 | FALSE |
| B_Coprococcus_catus | 216.8876 | 0 | 89 | 0 | 0.998029 | FALSE |
| B_Clostridium_spiroforme | 189.6883 | 0 | 77 | 0 | 0.997916 | FALSE |
| B_Erysipelatoclostridium_ramosum | 207.7867 | 0 | 75 | 0 | 0.997965 | FALSE |
| F_Candida.glabrata | 223.831 | 0 | 420 | 0 | 0.99897 | FALSE |
| F_Wickerhamomyces.ciferrii | 198.3768 | 0 | 138 | 0 | 0.998166 | FALSE |
| F_Saccharomyces.cerevisiae | 145.3036 | 0 | 56 | 0 | 0.997877 | FALSE |
| F_Candida.dubliniensis | 214.3 | 0 | 40 | 0 | 0.997761 | FALSE |
| F_Aspergillus.niger | 201.4 | 0 | 35 | 0 | 0.997691 | FALSE |
| V_Bacillus_phage_Basilisk | 237.4959 | 0 | 484 | 0 | 0.999145 | FALSE |
| V_Streptococcus_phage_Javan575 | 238.2832 | 0 | 452 | 0 | 0.999057 | FALSE |
| V_Streptococcus_phage_Javan235 | 233.6913 | 0 | 447 | 0 | 0.999037 | FALSE |
| V_Escherichia_virus_122 | 222.4083 | 0 | 436 | 0 | 0.999003 | FALSE |
| V_Gordonia_phage_Stormageddon | 239.2284 | 0 | 416 | 0 | 0.998967 | FALSE |
| V_Xanthomonas_phage_OP2 | 217.0807 | 0 | 409 | 0 | 0.998928 | FALSE |
| V_Clostridium_phage_phiMMP04 | 230.0873 | 0 | 401 | 0 | 0.998918 | FALSE |
| V_Paenibacillus_phage_phiERICV | 238.1407 | 0 | 398 | 0 | 0.998915 | FALSE |
| V_Streptococcus_satel- | 231.0955 | 0 | 398 | 0 | 0.998908 | FALSE |
| lite_phage_Javan593 | | | | | | |
| V_Clostridium_phage_PhiS63 | 247.7323 | 0 | 396 | 0 | 0.998908 | FALSE |

| | | | | Topo- |
|---|---|---|---|---|
| | | | | logical |
| | Self- | shared | | Coeffi- |
| | Loops | name | Stress | cient |
| Cluster 1 | | | | |
| B_Actinomyces_graevenitzii | 0 | B_Actinomyces_graevenitzii | 22664 | 0.085496 |
| B_Actinomyces_odontolyticus | 0 | B Actinomyces_odontolyticus | 18120 | 0.100294 |
| B_Bacteroides_dorei | 0 | B_Bacteroides_dorei | 18130 | 0.115326 |
| B_Bifidobacterium_dentium | 0 | B Bifidobacterium_dentium | 14102 | 0.11641 |
| B_Clostridium_spiroforme | 0 | B_Clostridium_disporicum | 90468 | 0.201379 |
| B_Clostridium_symbiosum | 0 | B_Clostridium_symbiosum | 40102 | 0.142076 |
| B_Enterococcus_faecalis | 0 | B Enterococcus_faecalis | 21108 | 0.098981 |
| B_Fusobacterium_ulcerans | 0 | B_Fusobacterium_ulcerans | 107602 | 0.185056 |
| B_Klebsiella_pneumoniae | 0 | B_Klebsiella_pneumoniae | 24354 | 0.113159 |
| B_Klebsiella_variicola | 0 | B_Klebsiella_variicola | 21990 | 0.104866 |
| F_Candida.albicans | 0 | F_Candida.albicans | 2330 | 0.143945 |
| F_Grosmannia.clavigera | 0 | F_Grosmannia.clavigera | 2590 | 0.144468 |
| F_Moesziomyces.antarcticus | 0 | F_Moesziomyces.antarcticus | 8922 | 0.12974 |
| F_Saccharomyces.cerevisiae | 0 | F_Saccharomyces.cerevisiae | 3864 | 0.122487 |
| F_Sordaria.macrospora | 0 | F_Sordaria.macrospora | 7954 | 0.122056 |
| V_Acinetobacter_phage_Ab105.3phi | 0 | V_Acinetobacter_phage_Ab105.3phi | 399232 | 0.129419 |
| V_Aeromonas_phage_Asfd_1 | 0 | V_Aeromonas_phage_Asfd_1 | 225354 | 0.140879 |
| V_Bacillus_phage_0305phi8.36 | 0 | V_Bacillus_phage_0305phi8.36 | 429814 | 0.125232 |
| V_Bacillus_phage_proCM3 | 0 | V_Bacillus_phage_proCM3 | 432166 | 0.125302 |
| V_Clostridium_phage_c.st | 0 | V_Clostridium_phage_c.st | 308512 | 0.128264 |
| V_Hokovirus_HKV1 | 0 | V Hokovirus_HKV1 | 519720 | 0.116206 |
| V_Klebsiella_phage_ST11.OXA245phi3.2 | 0 | V_Klebsiella_phage_ST11.OXA245phi3.2 | 366712 | 0.120668 |
| V_Klebsiella_phage_ST11.VIM1phi8.2 | 0 | V_Klebsiella_phage_ST11.VIM1phi8.2 | 234724 | 0.137011 |
| V_Klebsiella_phage_ST13.OXA48phi12.1 | 0 | V_Klebsiella_phage_ST13.OXA48phi12.1 | 348034 | 0.120813 |
| V_Prokaryotic_dsDNA_virus_sp. | 0 | V_Prokaryotic_dsDNA_virus_sp. | 318874 | 0.12797 |
| Cluster 2 | | | | |
| B_Megamonas_hypermegale | 0 | B_Megamonas_hypermegale | 170208 | 0.268794 |
| B_Megamonas_funiformis | 0 | B_Megamonas_funiformis | 137982 | 0.265438 |
| B_Asaccharobacter_celatus | 0 | B_Asaccharobacter_celatus | 55150 | 0.263285 |
| B_Ruminococcus bromii | 0 | B Ruminococcus bromii | 17062 | 0.287078 |
| B_Blautia_wexlerae | 0 | B_Blautia_wexlerae | 24262 | 0.290024 |
| B_Paraprevotella clara | 0 | B_Paraprevotella clara | 14352 | 0.255551 |
| B_Bacteroides_cellulosilyticus | 0 | B Bacteroides_cellulosilyticus | 17554 | 0.261562 |
| B_Coprococcus_catus | 0 | B_Coprococcus_catus | 10974 | 0.289957 |
| B_Clostridium_spiroforme | 0 | B_Clostridium_spiroforme | 20462 | 0.265634 |
| B_Erysipelatoclostridium_ramosum | 0 | B_Erysipelatoclostridium_ramosum | 23378 | 0.281936 |
| F_Candida.glabrata | 0 | F_Wickerhamomyces.ciferrii | 520300 | 0.28696 |
| F_Wickerhamomyces.ciferrii | 0 | F_Candida.glabrata | 32006 | 0.263799 |
| F_Saccharomyces.cerevisiae | 0 | F_Saccharomyces.cerevisiae | 74430 | 0.201506 |
| F_Candida.dubliniensis | 0 | F_Candida.dubliniensis | 1004 | 0.30879 |
| F_Aspergillus.niger | 0 | F_Aspergillus.niger | 10972 | 0.295742 |
| V_Bacillus_phage_Basilisk | 0 | V_Bacillus_phage Basilisk | 190388 | 0.302543 |
| V_Streptococcus_phage_Javan575 | 0 | V_Streptococcus_phage_Javan575 | 167568 | 0.304321 |
| V_Streptococcus_phage_Javan235 | 0 | V_Streptococcus_phage_Javan235 | 190254 | 0.299986 |
| V_Escherichia_virus_122 | 0 | V_Escherichia_virus_122 | 180506 | 0.285872 |
| V_Gordonia_phage_Stormageddon | 0 | V_Gordonia_phage_Stormageddon | 171200 | 0.305138 |
| V_Xanthomonas_phage_OP2 | 0 | V_Xanthomonas_phage_OP2 | 148928 | 0.279743 |
| V_Clostridium_phage_phiMMP04 | 0 | V_Clostridium_phage_phiMMP04 | 162078 | 0.294984 |
| V_Paenibacillus_phage_phiERICV | 0 | V_Paenibacillus_phage_phiERICV | 116090 | 0.304528 |
| V_Streptococcus_satel- | 0 | V_Streptococcus_satel- | 197602 | 0.296657 |
| lite_phage_Javan593 | | lite_phage_Javan593 | | |
| V_Clostridium_phage_PhiS63 | 0 | V_Clostridium_phage_Phis63 | 101846 | 0.317199 |

SUPPLEMENTARY TABLE 8
Comparison of key microorganisms in the core network between two clusters

| | | Cluster 1 | | Cluster 2 | | |
|---|---|---|---|---|---|---|
| | | average relative | | average relative | | |
| | | abundance (%) | prevalence | abundance (%) | Prevalence | p_relative_abundance |
| | Core species in Cluster 1 | | | | | |
| Bacteria | Klebsiella variicola | 0.407101905 | 30.16% | 0.042470571 | 17.14% | 0.343 |
| | Klebsiella pneumoniae | 1.044333016 | 34.92% | 0.090782714 | 27.14% | 0.118 |
| | Fusobacterium ulcerans | 0.021945238 | 11.11% | 0.002021286 | 4.29% | 0.143 |
| | Enterococcus faecalis | 0.042752857 | 12.70% | 0.022138429 | 7.14% | 0.407 |
| | Clostridium symbiosum | 0.082984603 | 41.27% | 0.076601714 | 40.00% | 0.858 |
| | Bifidobacterium dentium | 0.752015556 | 25.40% | 0.016494143 | 18.57% | 0.207 |
| | Bacteroides dorei | 2.48331873 | 33.33% | 2.291270286 | 45.71% | 0.853 |
| | Actinomyces odontolyticus | 0.06789619 | 69.84% | 0.142193571 | 72.86% | 0.471 |
| | Actinomyces graevenitzii | 0.024029048 | 42.86% | 0.092364571 | 50.00% | 0.356 |
| | Clostridium spiroforme | 0.047805714 | 88.89% | 0.020314714 | 25.71% | 0.323 |
| Fungi | Sordaria macrospora | 5.15340225 | 30.16% | 0.964887276 | 5.71% | 0.009 |
| | Saccharomyces cerevisiae | 16.40723517 | 22.86% | 2.153834636 | 7.94% | 0.002 |
| | Moesziomyces antarcticus | 1.443491188 | 9.52% | 0.115410599 | 1.43% | 0.028 |
| | Grosmannia clavigera | 9.337052862 | 27.14% | 0.389267353 | 6.35% | 0.002 |
| | Candida albicans | 6.344914367 | 28.57% | 5.337365065 | 14.29% | 0.011 |
| Virus | Prokaryotic_dsDNA_virus_sp. | 1.962471594 | 96.83% | 1.509620326 | 87.14% | 0.002 |
| | Klebsiella_phage_ST13.OXA48phi12.1 | 0.678541781 | 96.83% | 0.515338511 | 88.57% | 0.001 |
| | Klebsiella_phage_ST11.VIM1phi8.2 | 0.629786723 | 96.83% | 0.49620601 | 100.00% | 0.014 |
| | Klebsiella_phage_ST11.OXA245phi3.2 | 1.275642072 | 96.83% | 1.037695426 | 100.00% | 0.015 |
| | Hokovirus_HKV1 | 0.856623986 | 96.83% | 0.64875089 | 87.14% | 0.001 |
| | Clostridium_phage_c.st | 0.377647087 | 96.83% | 0.323657244 | 100.00% | 0.149 |
| | Bacillus_phage_proCM3 | 1.511168335 | 96.83% | 1.169324285 | 91.43% | 0.002 |
| | Bacillus_phage_0305phi8.36 | 0.627551732 | 96.83% | 0.528815515 | 100.00% | 0.054 |
| | Aeromonas_phage_Asfd_1 | 0.169080051 | 96.83% | 0.134027191 | 100.00% | 0.047 |
| | Acinetobacter_phage_Ab105.3phi | 0.73990191 | 96.83% | 0.596443699 | 100.00% | 0.012 |
| | Core species in Cluster 2 | | | | | |
| Bacteria | Paraprevotella_clara | 0.015213968 | 7.94% | 0.015501 | 12.86% | 0.983 |
| | Megamonas_hypermegale | 0.074568571 | 15.87% | 0.076883143 | 24.29% | 0.959 |
| | Megamonas_funiformis | 0.520759841 | 15.87% | 0.181513857 | 22.86% | 0.341 |
| | Erysipelatoclostridium_ramosum | 0.167201905 | 38.10% | 0.261386571 | 48.57% | 0.429 |
| | Coprococcus_catus | 0.148371587 | 39.68% | 0.293595286 | 54.29% | 0.052 |
| | Clostridium_spiroforme | 0.047805714 | 88.89% | 0.020314714 | 25.71% | 0.323 |
| | Blautia_wexlerae | 1.413865873 | 76.19% | 3.818035429 | 97.14% | 0.002 |
| | Bacteroides_cellulosilyticus | 0.128014286 | 47.62% | 0.280017714 | 45.71% | 0.139 |
| | Asaccharobacter_celatus | 0.031917302 | 39.68% | 0.071444143 | 47.14% | 0.099 |
| | Ruminococcus_bromii | 2.074178889 | 38.10% | 1.892165429 | 32.86% | 0.816 |
| Fungi | Wickerhamomyces.ciferrii | 10.38902467 | 27.14% | 51.22696122 | 68.25% | 0.000 |
| | Saccharomyces.cerevisiae | 16.40723517 | 21.43% | 2.153834636 | 7.94% | 0.002 |
| | Candida.glabrata | 14.87878112 | 65.71% | 0.933883255 | 41.27% | 0.000 |
| | Candida.dubliniensis | 1.711752288 | 18.57% | 0.004315529 | 4.76% | 0.261 |
| | Aspergillus.niger | 6.622653125 | 45.71% | 1.657805046 | 25.40% | 0.066 |
| Virus | Xanthomonas_phage_OP2 | 0.017749842 | 96.83% | 0.014510103 | 97.14% | 0.210 |
| | Streptococcus_satellite_phage_Javan593 | 0.025426986 | 96.83% | 0.015447558 | 95.71% | 0.042 |
| | Streptococcus_phage_Javan575 | 0.056341377 | 96.83% | 0.015368446 | 88.57% | 0.256 |
| | Streptococcus_phage_Javan235 | 0.020297669 | 98.41% | 0.014119589 | 92.86% | 0.035 |
| | Paenibacillus_phage_phiERICV | 0.019846728 | 96.83% | 0.014376806 | 92.86% | 0.015 |
| | Gordonia_phage_Stormageddon | 0.018604131 | 96.83% | 0.014432522 | 97.14% | 0.241 |
| | Escherichia_virus_122 | 0.015174003 | 98.41% | 0.014263944 | 95.71% | 0.735 |
| | Clostridium_phage_PhiS63 | 0.017675377 | 96.83% | 0.01483248 | 90.00% | 0.221 |
| | Clostridium_phage_phiMMP04 | 0.020053609 | 98.41% | 0.013409542 | 98.57% | 0.024 |
| | Bacillus_phage_Basilisk | 0.019482903 | 96.83% | 0.014350922 | 100.00% | 0.037 |

Supplementary Table 9 Questionnaire used for
post-acute COVID-19 symptom assessment

| | | |
|---|---|---|
| Symptoms | Month 3 | Month 6 |

Fever
Chills
Cough
Sputum Production
Sore throat
Congested or runny nose
Fatigue
Joint pain
Muscle pain
Shortness of breath
Headache
Dizziness
Nausea
Vomiting
Diarrhoea
Loss of taste
Loss of smell
Abdominal pain
Epigastric pain
Difficulty in concentration
Inability to exercise
Difficulty in sleeping
Anxiety
Sadness
Memory problem
Chest pain
Palpitations
Night sweats
Hair loss
Blurred vision
Any other symptoms

1. A method for determining a patient has or is at risk of severe COVID-19 or post-acute COVID-19 syndrome (PACS), comprising
(1) obtaining a set of training data by determining in fecal samples the relative abundance of the bacteria, viral, and fungi species listed in Table 3 and the clinical factors listed in Table 3 obtained from a cohort of subjects with severe COVID-19 or PACS and a cohort of subjects without severe COVID-19 or PACS;
(2) determining the relative abundance of the species and clinical factors listed in Table 3 in the patient;
(3) comparing the relative abundance of the species and clinical factors listed in Table 3 obtained from step (2) from the patient with the training data using random forest model, wherein decision trees are generated by random forest from the training data, and wherein the relative abundance of the species and clinical factors listed in Table 3 obtained in step (2) from the patient are run down the decision trees to generate a risk score; and
(4) determining the patient as having or at increased risk for severe COVID-19 or PACS when the risk score is greater than 0.5, and determining the patient as not having or at no increased risk for severe COVID-19 or PACS when the risk score is no greater than 0.5.

2. The method of claim 1, wherein the patient has been diagnosed with COVID.

3. The method of claim 1, wherein the patient has not been diagnosed with COVID.

4. The method of claim 1, wherein steps (1) and (2) each comprises determining the level of a DNA, RNA, or protein unique to one or more of the bacterial, viral, or fungal species set forth in Table 3.

5. The method of claim 1, wherein steps (1) and (2) each comprises metagenomics sequencing.

6. method of claim 1, wherein steps (1) and (2) each comprises a polymerase chain reaction (PCR).

7. The method of claim 6, wherein the PCR is quantitative PCR (qPCR).

8. The method of claim 1, further comprising treating the patient who has been determined as having or at increased risk for severe COVID-19 or PACS to prevent or alleviate symptoms of severe COVID-19 or PACS.

9. The method of claim 8, wherein the treating comprising administering to the patient a composition comprising an effective amount of (a) Bifidobacterium adolescentis or Faecalibacterium prausnitzii, or (b) an inhibitor specifically suppressing Ruminococcus gnavus, Klebsiella species (Klebsiella quasipneumonia, Klebsiella pneumoniae, and Klebsiella variicola), Clostridum species (Clostridum bolteae and Clostridium innocuum and Clostridium spiroforme); Asperigillus flavus, Candida glabrata, Candida albucans; Mycobacterium phage MyraDee, Pseudomonas virus Pf1, or Klebsiella phage.

10. The method of claim 9, wherein the treating comprises fecal microbiota transplantation (FMT).

11. The method of claim 10, wherein the FMT comprises delivery to the small intestine, ileum, or large intestine of the patient a composition comprising processed donor fecal material.

12. The method of claim 9, wherein the composition is formulated for oral administration.

13. The method of claim 12, wherein the composition is in the form of a food or beverage item.

14. method of claim 9, wherein the composition is formulated for direct deposit to the patient's gastrointestinal tract.

15. A method for predicting virus shedding duration in a COVID-19 patient, comprising:
(1) obtaining a set of training data by determining in fecal samples the relative abundance of species and clinical factors listed in Table 4 in a cohort of subjects who have been diagnosed with COVID-19 and had their SARS-CoV-2 viral shedding duration determined;
(2) determining the relative abundance of the species and clinical factors listed in Table 4 in the COVID-19 patient;
(3) comparing the relative abundance of species and clinical factors listed in Table 4 in the subject with the training data using random forest model; and
(4) generating viral shedding duration by the random forest model.

16. The method of claim 15, wherein steps (1) and (2) each comprises determining the level of a DNA, RNA, or protein unique to one or more of the bacterial species set forth in Table 4.

17. The method of claim 15, wherein steps (1) and (2) each comprises metagenomics sequencing.

18. The method of claim 15, wherein steps (1) and (2) each comprises a polymerase chain reaction (PCR).

19. The method of claim 18, wherein the PCR is quantitative PCR (qPCR).

20. The method of claim 15, further comprising keeping the patient in isolation for the viral shedding duration determined in step (4).
